SEQUENCE LISTING (2 sequences)

SEQ ID NO: 1 - DNA, 20 bp, Synthetic construct
  source 1..20: mol_type = other DNA
gcgtggagccgcgataaccc

SEQ ID NO: 2 - DNA, 19 bp, Synthetic construct
  source 1..19: mol_type = other DNA
cgcacgtgtgccccgcgta

(19) United States
(12) Patent Application Publication
(10) Pub. No.: US 2025/0313807 A1
(43) Pub. Date: Oct. 9, 2025

(54) SYSTEMS AND METHODS FOR CHARACTERIZATION OF POLYCYSTIC KIDNEY DISEASE

(71) Applicant: University of Washington, Seattle, WA (US)

(72) Inventors: Benjamin Freedman, Seattle, WA (US); Jonathan Himmelfarb, Seattle, WA (US)

(73) Assignee: University of Washington, Seattle, WA (US)

(21) Appl. No.: 18/864,983

(22) PCT Filed: May 24, 2023
(86) PCT No.: PCT/US2023/067399

§ 371 Date: Nov. 12, 2024

Related U.S. Application Data
(60) Provisional application No. 63/346,279, filed on May 26, 2022.

(51) Int. Cl. C12N 5/071; C12M 3/00; C12M 3/06

(52) U.S. Cl. CPC C12N 5/0686; C12M 21/08; C12M 23/16; C12N 2503/04; C12N 2510/00

(57) ABSTRACT

Microfluidic systems, kits, and methods for characterization of polycystic kidney disease (PKD) are described. In an embodiment, the microfluidic system includes a flow device comprising an inlet, an outlet, and a channel comprising a functionalized site configured for cell culture. In an embodiment, a genetically modified (GM) human kidney organoid is cultured at the functionalized site, optionally in the presence of a fluidic flow, to produce PKD cysts for use as a model system for characterization of mechanisms of PKD onset, progression, diagnosis, and response to treatment.

### CROSS-REFERENCE TO RELATED APPLICATION(S)

This application claims the benefit of U.S. Provisional Application No. 63/346,279 filed May 26, 2022. The content of the above-referenced application is hereby incorporated by reference in its entirely for all purposes.

### STATEMENT OF GOVERNMENT LICENSE RIGHTS

This invention was made with government support under Grant No. R01DK117914, awarded by the National Institute of Diabetes and Digestive and Kidney Diseases (NIDDK) [NIH] and Grant Nos. K01DK102826 and UG3TR002158 and UG3TR003288, awarded by the National Institutes of Health (NIH). The government has certain rights in the invention.

### STATEMENT REGARDING SEQUENCE LISTING

The sequence listing associated with this application is provided in XML format in lieu of a paper copy and is hereby incorporated by reference into the specification. The name of the XML file containing the sequence listing is 3915-P1305WO.UW_SequenceListing.xml. The XML file is 3 KB; was created on May 4, 2023, and is being submitted via Patent Center with the filing of the specification.

### BACKGROUND

Autosomal dominant polycystic kidney disease (PKD) is commonly inherited as a heterozygous, loss-of-function mutation in either PKD1 or PKD2, which encode the proteins polycystin-1 (PC1) or polycystin-2 (PC2), respectively. PKD is characterized by the growth of large, fluid-filled cysts from tubules or ductal structures in kidneys and other organs and is among the most common life-threatening monogenic diseases and kidney disorders. At the molecular level, PC1 and PC2 form a receptor-channel complex at the primary cilium that is poorly understood but possibly acts as a flow-sensitive mechanosensor. Loss of this complex results in the gradual expansion and dedifferentiation of the tubular epithelium, including increased proliferation and altered transporter expression and localization.

PKD treatments show potential, but their discovery and use is limited at least in part due to limited knowledge of mechanisms of PKD onset and progression. Accordingly, there is a need for an improved understanding of mechanisms of PKD for development of improved treatments. Since mechanisms of PKD are difficult to decipher in vivo, and murine models do not fully phenocopy or genocopy the human disease, there is a need for an improved human model of PKD for in vitro use that recreates microenvironments involved with PKD onset and progression. The present disclosure addresses these and other long-felt and unmet needs.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In an aspect, the disclosure provides a method for characterizing polycystic kidney disease (PKD) in vitro, the method comprising: culturing a genetically modified (GM) human kidney organoid within a flow device comprising a channel with a functionalized site configured for cell culture and an inlet and an outlet configured for flow of a fluid at the functionalized site for a PKD cyst; contacting the PKD cyst with the fluid having a property; and measuring a response of the PKD cyst to the property of the fluid. The property of the fluid can include any property of the fluid, such as a physical property (e.g., pressure, volume, flow rate, temperature) or a chemical property (e.g., solute identity and concentration). The flow of the fluid through the channel generally approximates the physiological flow of fluid within the kidney microenvironment, providing an improved model system for studying the PKD disease state.

In embodiments, the human kidney organoid has a genetic modification that is associated with PKD. This genetic modification can be implemented to cause a PKD phenotype, having increased PKD-specific cysts compared to the unmodified condition, when the GM human kidney organoid is cultured. The system and method can reliably and reproducibly form PKD cysts for further study of the PKD disease state.

In embodiments of the method for characterizing PKD in vitro, the method further comprises: creating a PKD genotype in a human stem cell. and differentiating the human stem cell into the GM human kidney organoid. Genetic engineering techniques, such as CRISPR-based techniques, can be used to knock down, knock out, or otherwise deactivate one or more genes to produce the PKD genotype.

In embodiments of the method for characterizing PKD in vitro, the method further comprises: genetically deactivating a PKD2 gene in the human stem cell and/or the GM human kidney organoid to produce a PKD2 negative human stem cell and/or a PKD2 negative GM human kidney organoid; and/or genetically deactivating a PKD1 gene in the human stem cell and/or the GM human kidney organoid to produce a PKD1 negative human stem cell and/or a PKD1 negative GM human kidney organoid. Mutations to either or both of PKD1 and PKD2 can be used to produce induced pluripotent stem (iPS) cells that have the potential to form human kidney organoids with an inclination to form PKD cysts. The organoids can be produced through differentiation according to organoid culture techniques.

In embodiments of the method for characterizing PKD in vitro, the characterization comprises: determining a mechanism of PKD cyst formation, expansion, and/or contraction in response to the property of the fluid. The mechanism can include a biomolecular mechanism, a macromolecular mechanism, a molecular mechanism, or another mechanism involved with PKD cyst formation, expansion, and/or contraction. The mechanism can relate to how PKD progresses and/or how PKD responds to a treatment or therapy.

In embodiments of the method for characterizing PKD in vitro, the method further comprises: determining whether the PKD cyst absorbs glucose; determining whether glucose absorption increases PKD cyst formation; and/or determining a polarization of the PKD cyst. The polarization of the PKD cyst may be inverted (apical surface facing outwards towards the media), for example.

In embodiments of the method for characterizing PKD in vitro, the method further comprises: controlling a volume, a solute concentration, and/or a flow rate of the fluid as the property of the fluid; and determining the response of the PKD cyst to the volume, the solute concentration, and/or the flow rate of the fluid. These or other properties of the fluid can be controlled to mimic the kidney microenvironment and determine how the PKD cyst responds to the changing model system microenvironment.

In embodiments of the method for characterizing PKD in vitro, a flow of the fluid corresponds with formation and/or expansion of the PKD cyst. It is described herein that fluid flow increases formation of the PKD cyst; therefore, this response can be used as part of a method for generating the PKD cyst for further study, as explained in more detail elsewhere herein.

In embodiments of the method for characterizing PKD in vitro, the inlet is fluidly connected to the outlet and the fluid flows from the inlet through the channel toward the outlet according to the flow rate and flows over the PKD cyst of the GM human kidney organoid. Since the system closely models fluidic flow in the kidney microenvironment, alterations to the model system can be used to model changes that might occur in the body, for example, in therapy and/or non-therapy scenarios.

In embodiments of the method for characterizing PKD in vitro, the method further comprises: contacting the PKD cyst with a glucose transport inhibitor to determine an effect of glucose transport inhibition on PKD cyst formation, expansion, and/or contraction. The presence of the glucose transport inhibitor in the fluid impacts PKD cyst formation, for example.

In embodiments of the method for characterizing PKD in vitro, the glucose transport inhibitor comprises phloretin, phloridzin, and/or dapagliflozin. Inclusion of one or more of these or other glucose transport inhibitors can be implemented to determine whether glucose transport is associated with PKD cyst formation.

In another aspect, the disclosure provides a method for formation of a PKD cyst of a GM human kidney organoid for characterizing PKD in vitro, the method comprising: culturing the GM human kidney organoid within a flow device having a channel with a functionalized site configured for cell culture and an inlet and an outlet configured for flow of a fluid at the functionalized site; and contacting the PKD cyst with a flow of the fluid. The flow of the fluid increases formation of PKD cysts within the model system.

In embodiments of the method for formation of a PKD cyst, the human kidney organoid has a genetic modification that is associated with PKD. Any genetic modification can be implemented to cause a PKD phenotype, having PKD cysts, when the GM human kidney organoid is cultured. The system and method can reliably and reproducibly form PKD cysts for further study of the PKD disease state.

In embodiments of the method for formation of a PKD cyst, the method further comprises: contacting the PKD cyst with glucose to increase formation of the PKD cyst. Increased formation of the PKD cyst in the presence of glucose can be implemented in a method to produce PKD cysts for studying a mechanism of the PKD disease state.

In embodiments of the method for formation of a PKD cyst, the characterization comprises: determining a mechanism of PKD cyst formation, expansion, and/or contraction in response to the fluid. In instances where the PKD cyst is formed, a person of skill in the art can determine the mechanism responsible by varying the properties of the fluid and determining characteristics of formation of the PKD cyst as a result of the properties of the fluid.

In embodiments of the method for formation of a PKD cyst, the method further comprises: controlling a volume, a solute concentration, and/or a flow rate of the fluid as the property of the fluid; and determining the response of the PKD cyst to the volume, the solute concentration, and/or the flow rate of the fluid. These or other properties of the fluid can be controlled to mimic the kidney microenvironment and control how the PKD cyst responds to the changing model system microenvironment.

In embodiments of the method for formation of a PKD cyst, a flow of the fluid corresponds with formation of the PKD cyst. In such instances, the PKD cyst is more readily formed as a result of the flow of the fluid.

In embodiments of the method for formation of a PKD cyst, the inlet is fluidly connected to the outlet and the fluid flows from the inlet through the channel toward the outlet according to the flow rate and flows over the PKD cyst of the GM human kidney organoid. Since the system closely models fluidic flow in the kidney microenvironment, alterations to formation of the PKD cyst in the model system can be used to model changes that might occur in the body, for example, in therapy and/or non-therapy scenarios, that impact formation of a PKD cyst in the human body.

In another aspect, the disclosure provides a microfluidic system for characterization of PKD in vitro, the microfluidic system comprising: a flow device having a channel with a functionalized site configured for cell culture and an inlet and an outlet configured for flow of a fluid at the functionalized site, wherein the inlet is fluidly connected with the outlet via the channel. The system can receive fluid from a fluid pump or syringe pump, or another fluid source, for controlled fluid flow through the channel and across the functionalized site of the system. In this manner, the GM human kidney organoid, when present at the functionalized site, is reliably exposed to the fluid flow for formation of PKD cysts for use as a model system.

In embodiments of the microfluidic system, the microfluidic system further comprises a GM human pluripotent stem cell or a GM human kidney organoid configured for a PKD cyst and having a genetic modification that is associated with PKD. The GM human pluripotent stem cell or the GM human kidney organoid can be provided as a separate item of the system, and a user can inject or introduce the GM human pluripotent stem cell or the GM human kidney organoid into the microfluidic system for culture at the functionalized site for further use. In instances where the GM human pluripotent stem cell is provided, the user can differentiate the GM human pluripotent stem cell to form the GM human kidney organoid and can further culture the GM human kidney organoid, as described herein, to form the PKD cyst.

In embodiments of the microfluidic system, the genetic modification that is associated with PKD comprises a genetically deactivated PKD2 gene and/or a genetically deactivated PKD1 gene. The genetic modification can be preexisting with the GM human pluripotent stem cell and/or the GM human kidney organoid, for example, as a cryopreserved GM cell line provided as part of a kit for research or other use. The end user can then thaw and culture the cells without needing to genetically manipulate the cells beforehand. This enables scalable and reproducible research into the PKD disease state by research and medical communities.

In embodiments of the microfluidic system, the functionalized site comprises an extracellular matrix (ECM) for cell culture. An example ECM that is suitable for cell culture is Corning® Matrigel® Matrix.

In embodiments of the microfluidic system, the characterization comprises determination of a mechanism of PKD cyst formation, expansion, and/or contraction in response to the fluid. The mechanism can be associated with a determination of whether a sample, such as a cell or tissue sample from a patient, has PKD or has an inclination to form PKD cysts, for example, for diagnostic or other purposes. The mechanism can include a determination of whether a cell or tissue sample from a patient that forms cysts responds to treatment, such as an experimental treatment, for example.

### DESCRIPTION OF THE DRAWINGS

The foregoing aspects and many of the attendant advantages of the claimed subject matter will become more readily appreciated as the same become better understood by reference to the following detailed description, when taken in conjunction with the accompanying drawings, wherein:

FIG. 1A shows organoid PKD cysts expanding under flow, according to embodiments. Representative images of organoids on days following transfer to suspension culture (upper), with quantification (lower) of cyst incidence as a fraction of the total number of organoids (mean±s.e.m. from n≥4 independent experiments per condition; ****, p<0.0001).

FIG. 1B shows a schematic of a workflow for a fluidic condition with an example system or kit of the disclosure, according to embodiments.

FIG. 1C shows time-lapse phase contrast images of PKD organoids under flow (0.2 dynes/cm²), according to embodiments.

FIG. 1D shows average growth rates of control organoids (Ctrl org.), non-cystic compartments of PKD organoids (PKD org.), and cystic compartments of PKD organoids (PKD cysts) under flow (0.2 dynes/cm²), according to embodiments. Each experiment was performed for 6 hours. Cyst growth rate was calculated on an individual basis as the maximal size of the cyst during the time course, divided by the time point at which the cyst reached this size. (mean±s.e.m. from n≥4 independent experiments; each dot represents the average growth rate of organoids in a single experiment. ****, p<0.0001).

FIG. 2A shows rhodamine dextran (10 kDa) epifluorescence in static (non-diffusive), diffusive static, and fluidic conditions. ‘Lane’ indicates channel interior.

FIG. 2B shows volume can partially substitute for flow in cyst expansion with time lapse phase contrast images of cysts in these conditions.

FIG. 2C shows average growth rates (μm²/hr) of cysts in diffusive static condition with different volumes, compared to fluidic or non-diffusive static, according to embodiments. Each experiment was performed for 6 hours. Cyst growth rate was calculated on an individual basis as the maximal size of the cyst during the time course, divided by the time point at which the cyst reached this size. (n≥8 cysts (dots) pooled from two or more independent experiments; ***, p<0.05).

FIG. 2D shows a schematic of an experiment testing the effect of volume vs. pressure on cyst growth.

FIG. 2E shows representative phase contrast images and FIG. 2F shows quantification of growth rate of cysts suspended in either 0.5 or 10 mL of media under equivalent hydrostatic pressures (mean±s.e.m. of n≥14 cysts per condition pooled from three independent experiments; ***, p<0.05), according to embodiments.

FIG. 2G shows growth profiles of individual cysts (lines) over time in microfluidic devices from 0-5 hour. Measurements were made every 5 minutes using ImageJ software. The cyst area was normalized by dividing by the starting area.

FIG. 2H shows sum of squares values from linear regression models that were run on each individual cyst (n≥7 organoids per condition, pooled from 4 or more independent experiments; p=0.0342 versus diffusive static and 0.0411 versus static). Error bars, standard error.

FIG. 3A shows NBD-Glucose background levels in non-diffusive static, diffusive static, and fluidic conditions after 12 hours.

FIG. 3B shows PKD organoids absorbing glucose under fluidic and static conditions, according to embodiments. Representative phase contrast and wide field fluorescence images of organoids in diffusive static and fluidic conditions, five hours after introduction of NBD-Glucose. Arrows are drawn to indicate representative line scans.

FIG. 3C shows a line scan analysis of glucose absorption in PKD cysts under static and fluidic conditions after five hours (mean±s.e.m. from n 7 cysts per condition pooled from 3 independent experiments; each n indicates the average of 4 line scans taken from a single cyst). Background fluorescence levels were calculated at each timepoint by measuring the fluorescence intensity of a square region placed in the non-organoid region of the image.

FIG. 3D shows NBD-Glucose absorption in the non-cystic compartment of PKD organoid, for diffusive static 20 mL vs. 1 mL (110 μM NBD-Glucose, mean±s.e.m., n≥4 independent experiments), and FIG. 3E shows diffusive static 25 mL vs. fluidic (36.5 μM NBD-Glucose, n≥5 independent experiments).

FIG. 3F shows confocal fluorescence images of SGLT2 and ZO1 in PKD1 tubules.

FIG. 3G shows confocal fluorescent images of NBD-Glucose in organoid tubules, fixed and stained with fluorescent cell surface markers.

FIG. 3H shows representative time-lapse images of NBD-Glucose accumulation in a PKD organoid cyst, followed by washout into media containing unlabeled glucose after 24 hours, all performed under continuous flow, according to embodiments.

FIG. 4A shows PKD cysts expand in response to glucose stimulation, according to embodiments. The figure shows representative time lapse brightfield images and FIG. 4B shows quantification of change in cyst size in PKD organoids in static suspension cultures containing with D-Glucose concentrations (mean±s.e.m., n≥6 pooled from 4 independent experiments, each dot indicates a single cyst).

FIG. 4C shows representative time lapse images and FIG. 4D shows quantification of PKD organoids in 15 mM D-Glucose treated with phloretin (mean±s.e.m., n≥10 cysts pooled from 4 independent experiments, p=0.0231).

FIG. 4E shows quantification of maximum intensity projections of live dead staining in organoids treated with phloretin (mean±s.e.m., n≥11, pooled from 2 independent experiments, each dot indicates a cystic organoid).

FIG. 4F shows representative images of live staining with Calcein AM.

FIG. 4G shows brightfield images and FIG. 4H shows quantification of size changes in cystic PKD organoids in 15 mM D-Glucose treated with probenecid (mean±s.e.m., n≥9 pooled from 2 independent experiments).

FIG. 5A shows PKD cysts form via expansion of outwards-facing epithelium, according to embodiments. The figure shows confocal immunofluorescence images of cilia (AcT) and tight junctions (ZO-1) in proximal tubules (LTL) of PKD and non-PKD organoids, as well as in PKD cyst lining epithelial cells. Dashed arrow indicates how line scans were drawn.

FIG. 5B shows ZO1 and AcT intensity profiles in cysts vs. organoids. Line scans were drawn through cilia from lumen to exterior of structures. (mean±s.e.m. from n=5 line scans pooled from 3 organoids or cysts per condition).

FIG. 5C shows fluorescent images of stromal markers in PKD organoids compared to human kidney tissue from a middle-aged adult with autosomal dominant PKD. Scale bars 20 μm.

FIG. 5D shows fluorescent images of cysts after having been overlaid with collagen.

FIG. 5E shows representative Z-stack confocal images of early (day 30) PKD organoid cyst in adherent culture. Zoom shows boxed region. White arrow indicates a podocyte cluster continuous with the peripheral epithelium.

FIG. 5F shows a close-up image showing peripheral epithelium of control (non-PKD) organoid in adherent culture. Arrowhead indicates region of epithelial invagination.

FIG. 5G shows phase contrast time-lapse images showing formation of PKD cysts from non-cystic structures in adherent cultures. Arrows indicate tubular structures internal to the peripheral cyst.

FIG. 5H shows an example schematic model of absorptive cyst expansion in organoids, according to embodiments. Fluid flow 51 of fluid 52 is absorbed into outwards-facing proximal tubular epithelium 54, which generates internal pressure 53 that drives expansion and stretching of the epithelium 54. A simplified organoid 55 lacking podocytes or multiple nephron branches is shown for clarity.

FIG. 6A shows PKD cysts in vivo absorbing glucose into the surrounding interstitium. PAS stains of 2-month-old and 6-month-old Pkd1^{RC/RC} mice. Scale bars 50 μm.

FIG. 6B shows confocal images of stromal basement membrane (LAMA1) with cilia (AcT) or (as shown at FIG. 6C) endothelial cells (CD31) in Pkd1^{RC/RC} versus WT 2-month-old mice. Arrowheads indicate areas of detached or expanded interstitium surrounding the cyst.

FIG. 6D shows a schematic of a glucose uptake assay, according to embodiments.

FIG. 6E shows representative images and FIG. 6F shows a line scan analysis of PKD cysts after perfusion with fluorescent NBD-Glucose or unlabeled PBS control (mean±s.e.m., n≥17 cysts pooled from three mice per condition). Dashed magenta arrows indicate how line scans were drawn.

FIG. 7A shows a static condition that allows diffusion that promotes cyst expansion. Line drawings of food dye moving through ‘non-diffusive’ static condition incorporating a luer lock syringe and FIG. 7B shows a ‘diffusive’ static condition incorporating a media reservoir suspended over a microfluidic chip and connected by wide tubing.

FIG. 8A shows SGLT2 is expressed in PKD cysts and organoids with representative immunoblots and FIG. 8B shows quantification of SGLT2 levels in organoid cultures (mean±s.e.m. from three independent experiments).

FIG. 8C shows representative confocal images of SGLT2 and NBD-Glucose in PKO cysts, with zoom of boxed white region. Scale bars 100 m.

FIG. 9A shows glucose accumulates in organoids and cysts. The figure shows representative confocal images of organoids following 5 hr of exposure to NBD-Glucose. Gray and white dotted lines mark demonstrate tracing of non-cyst compartment and cyst, respectively.

FIG. 9B shows raw and FIG. 9C shows background subtracted NBD-Glucose fluorescence intensity over time in non-cyst compartments and cysts (mean±s.e.m. from seven organoids, pooled from three independent experiments; *, p<0.05).

FIG. 9D shows total glucose levels in these structures, calculated as (Area*Mean Intensity), with background subtraction based on 0 hr (mean±s.e.m. from seven organoids, pooled from three independent experiments; *, p<0.05).

FIG. 10A shows quantification of cyst size fold change for cysts treated with D-Glucose, showing all data points including outliers.

FIG. 10B shows representative time lapse phase contrast images of high (60 mM) versus standard (11 mM) glucose treatments.

FIG. 10C shows confocal images and FIG. 10D shows quantification of live/dead staining (calcein AM/propidium iodide 1:2000) in organoids with high or standard glucose levels, compared to 10% DMSO as a positive control for cytotoxicity (mean±s.e.m., n ˜14 cysts pooled from 4 independent experiments).

FIG. 11A shows quantification of average change in cyst size for cysts treated with D-Glucose, showing all data points including outliers.

FIG. 11B shows representative phase contrast time course images of organoids treated with phloretin or 10% DMSO.

FIG. 11C shows quantification of live/dead staining at 24 hours (n ˜7 organoids pooled from 3 independent experiments).

FIG. 11D shows quantification of cyst size and FIG. 11E shows live/dead ratio for organoids treated for 48 hours with phloridzin and dapagliflozin (mean±s.e.m., n ˜15 cysts per condition, pooled from 3 independent experiments).

FIG. 11F shows cyst size quantification of probenecid treatment (n ˜9 pooled from 2 independent experiments).

FIG. 12A shows organoid peripheral epithelium faces outwards and contains tubular infolds. The figure shows a full channel panel and FIG. 12B shows zoomed out confocal immunofluorescence images of control organoids, showing peripheral epithelium. White boxed region highlights the images shown in FIG. 5F.

FIG. 13A shows PKD organoid cystogenesis occurs via expansion of peripheral epithelium. FIGS. 13A and 13B show time lapse images of cysts forming from 12 representative PKD2^{-/-} (FIG. 13A) and PKD1^{-/-} (FIG. 13B) organoids, according to embodiments.

FIG. 14 shows an example schematic of absorptive cyst formation in kidney tissue, according to embodiments. Fluid (arrows) is absorbed through proximal tubules 143 into the underlying interstitium, which partially detaches from the epithelium. The tubules 143 then expand and deform to fill the interstitial space 144, reaching a low-energy conformation in which the withheld volume is ultimately transferred back into the luminal space 145 of the nascent microcyst. A simplified model is shown and represents one possible explanation of the findings, without wishing to be bound by any particular theory.

FIG. 15 shows a formula for calculating pressure in 1 mL versus 25 mL static conditions.

FIG. 16 shows a flow chart of an example method of use of a microfluidic system or kit of the disclosure for characterization of PKD with a PKD cyst of a genetically modified human kidney organoid, according to embodiments.

### DETAILED DESCRIPTION

Human kidney organoids can be derived from human pluripotent stem cells (hPSC), and contain podocyte, proximal tubule, and distal tubule segments in contiguous, nephron-like arrangements. Differentiation of these organoids is sensitive to the physical properties of the extracellular microenvironment. Organoids derived from gene-edited hPSC with biallelic, truncating mutations in PKD1 or PKD2 develop cysts from kidney tubules, reconstituting the phenotype of the disease. Culture of organoids under suspension conditions dramatically increases the expressivity of the PKD phenotype, revealing a critical role for microenvironment in cystogenesis. Fluid flow is a major feature of the nephron microenvironment and can potentially contribute to PKD, however, physiological rates of flow have not yet been achieved in kidney organoid cultures or PKD models.

Accordingly, the disclosure provides ‘kidney on a chip’ microphysiological systems and fit-for-purpose platforms integrating flow with kidney cells to model physiology and disease in a setting that more closely simulates the in vivo condition compared with other approaches, such as monolayer cultures. These kits and systems can be made and used according to various methods of the disclosure. The disclosure enables a person having skill in the art to effectively integrate organ on chip systems with organoids, which can be derived from hPSC as a renewable and gene-editable cell source. In addition, the disclosure provides an example investigation of the effect of fluid flow on a PKD cyst of a human organoid using an example system of the disclosure.

### Microfluidic Systems and Kits

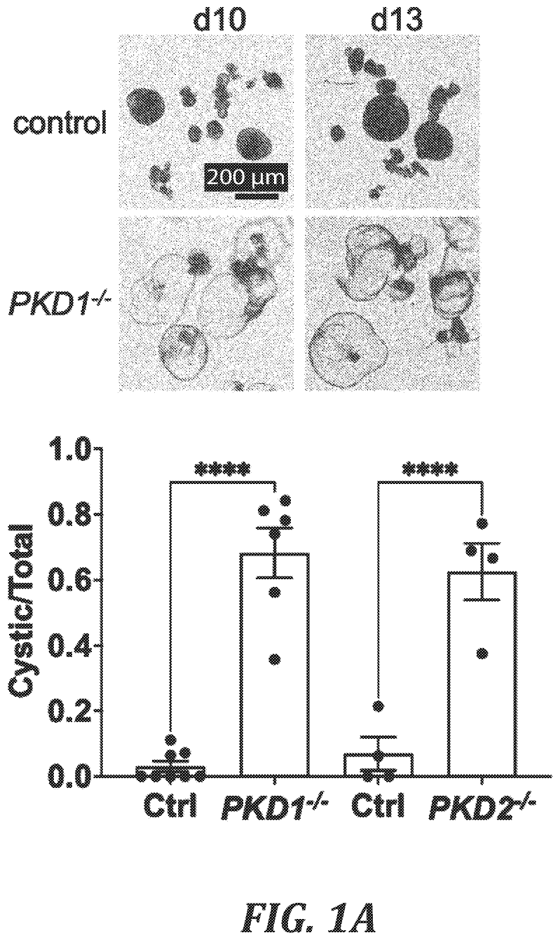

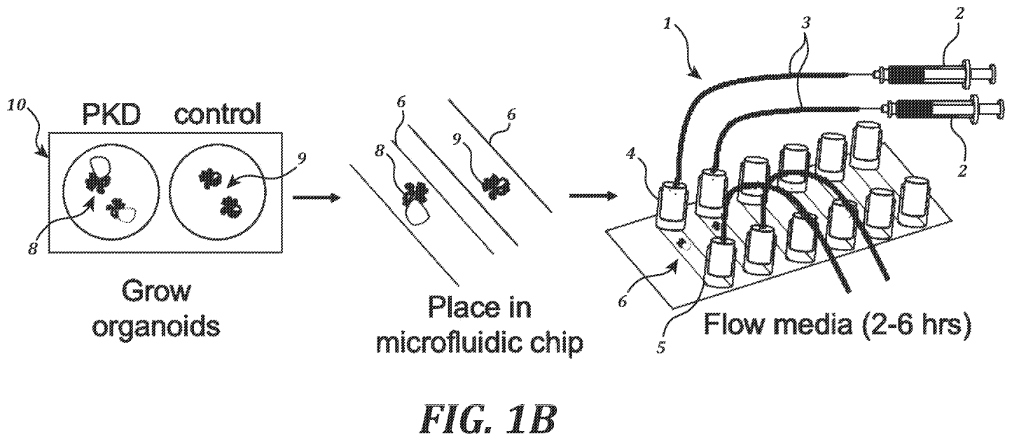

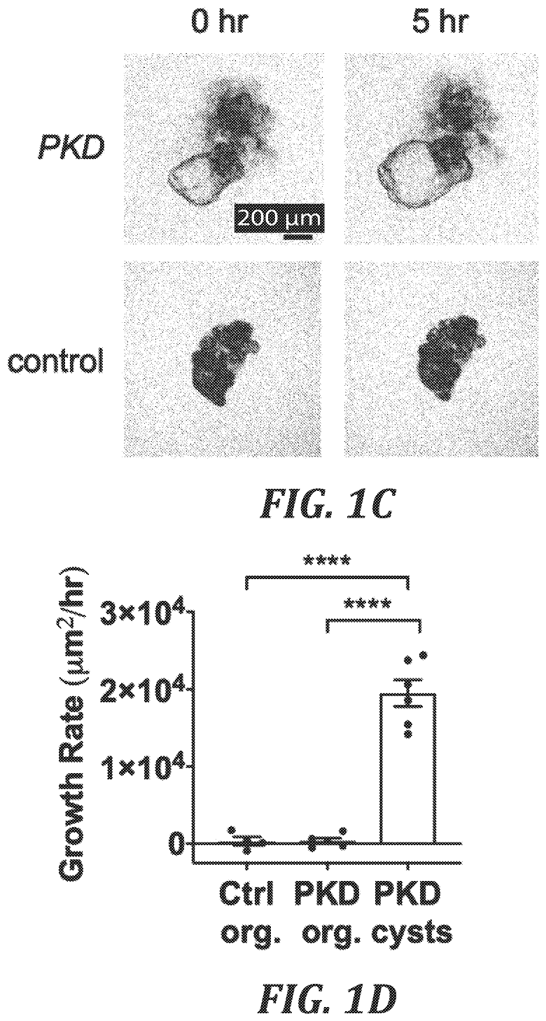

The disclosure provides an improved microfluidic system for characterization of PKD in vitro. An example microfluidic system 1 is shown at FIG. 1B. The microfluidic system 1 includes a flow device (2, 3, 4, 5, 6) comprising a channel 6 with a functionalized site configured for cell culture and an inlet 4 and an outlet 5 configured for flow of a fluid at the functionalized site, such that the inlet 4 is fluidly connected with the outlet 5 via the channel 6. In the shown embodiment, tubing 3 is included as an example structure for transport of fluid from syringe pump 2 to the inlet 4, but in other embodiments, other structures can be used for this purpose. System 1 is configured to receive fluid from a fluid pump or syringe pump 2, or another fluid source, for controlled fluid flow through channel 6 and across the functionalized site of system 1. In this manner, the genetically modified (GM) human kidney organoid, when present at the functionalized site, is reliably exposed to the fluid flow for formation of PKD cysts for use as a model system.

While the microfluidic system 1 is shown with a GM human kidney organoid 7 configured for a PKD cyst 8 and comprising a genetic modification that is associated with PKD, in other embodiments, the system can be provided without the GM human kidney organoid or a GM human pluripotent stem cell, and either or both of these components can be added to the system 1 while preparing the system 1 for use. Accordingly, in embodiments, the GM human pluripotent stem cell or the GM human kidney organoid can be provided as a separate item, optionally as a separate item of the system, and a user can inject or introduce the GM human pluripotent stem cell or the GM human kidney organoid into the microfluidic system for culture at the functionalized site for further use. In instances where the GM human pluripotent stem cell is provided, the user can differentiate the GM human pluripotent stem cell to form the GM human kidney organoid and can further culture the GM human kidney organoid, as described herein, to form the PKD cyst. As an example, PKD1^{-/-} or PKD2^{-/-} hPSC can be differentiated to form kidney organoids. Organoids can be purified by microdissection and transferred into gas-permeable, tissue culture-treated polymer flow chambers (e.g., flow devices), that are optically clear and large enough to comfortably accommodate organoids and cysts. Organoids can then be subjected to fluid flow with a wall shear stress of 0.2 dynes/cm², which approximates physiological shear stress within kidney tubules, to cause PKD cysts in PKD organoids to increase in size rapidly under the flow.

In embodiments of the microfluidic system 1, the genetic modification that is associated with PKD comprises a genetically deactivated PKD2 gene and/or a genetically deactivated PKD1 gene. The genetic modification can be provided to the user as preexisting with the GM human pluripotent stem cell and/or the GM human kidney organoid, for example, as a cryopreserved GM cell line provided as part of a kit for research, diagnostic, or other use. The cryopreserved GM cell line can be thawed and cultured by a user without the user needing to genetically manipulate the cells beforehand, and then transferred into the channel of the microfluidic system, optionally by way of an access port of the microfluidic system 1. However, in other embodiments, the GM human pluripotent stem cell and/or the GM human kidney organoid can be provided to the user with the cells already within the channel 6, at or near the functionalized site, and the user does not need to culture and then transfer the cells from a separate culture container to the functionalized site of the channel 6 of the microfluidic system 1. In this manner, system 1 is easier to use and requires fewer steps to get started with using the model system to investigate mechanisms of PKD disease. The flexibility in implementation also enables scalable and reproducible research into the PKD disease state, for example, by medical researchers.

In embodiments of the microfluidic system, the functionalized site comprises an extracellular matrix (ECM) for cell culture. An example ECM that is suitable for cell culture is Corning® Matrigel® Matrix.

In embodiments of the microfluidic system 1, the characterization comprises determination of a mechanism of PKD cyst formation, expansion, and/or contraction in response to the fluid. The mechanism can be associated with a determination of whether a sample, such as a cell or tissue sample from a patient, has PKD or has an inclination to form PKD cysts, for example, for research, medical, diagnostic, or other purposes. In embodiments, a kit or system can be used for determination of whether a cell or tissue sample from a PKD patient responds to treatment, such as an experimental treatment, for example.

In embodiments, the microfluidic system 1 is provided as a kit, for example, to a user. The kit can include the microfluidic system 1 in combination with cells such as GM stem cells and/or GM human kidney organoids, instructions, e.g., how to culture cells, culture media, tablets for formulating culture media, microscope cover slips for imaging the GM human kidney organoids in culture, and the like. In this manner, the kit can include a plurality of essential or helpful components for culturing GM human kidney organoids within the microfluidic system 1.

### Methods

The disclosure also provides methods of use of systems and kits as described herein. The systems and kits can be used for characterizing PKD in vitro; such a method comprises culturing a GM human kidney organoid within a flow device having a channel with a functionalized site configured for cell culture and an inlet and an outlet configured for flow of a fluid at the functionalized site for a PKD cyst; contacting the PKD cyst with the fluid having a property; and measuring a response of the PKD cyst to the property of the fluid. The systems and kits enable researchers, technicians, and clinicians, and others in the art, to investigate mechanisms of the PKD disease state.

In embodiments, the property of the fluid can include any property of the fluid, such as a physical property (e.g., pressure, volume, flow rate, temperature) or a chemical property (e.g., solute identity and concentration; e.g., glucose at a concentration) that is controlled or controllable by a user or researcher. The flow of the fluid through the channel approximates the physiological flow of fluid within the kidney microenvironment, providing an improved model system for studying the PKD disease state.

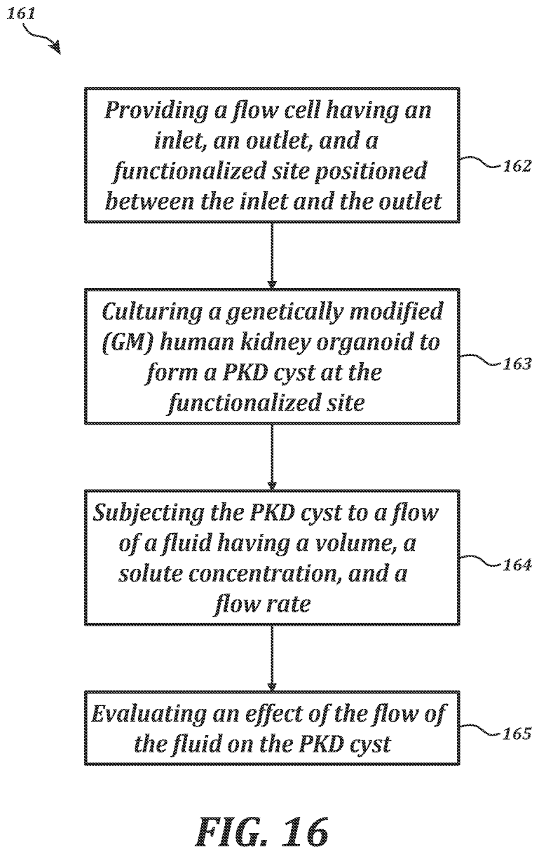

In embodiments, an example method 161 as shown at FIG. 16 is implemented. The example method 161 comprises, at step 162, providing a flow cell having an inlet, an outlet, and a functionalized site positioned between the inlet and the outlet. The example method 161 further comprises, at step 163, culturing a genetically modified (GM) human kidney organoid to form a PKD cyst at the functionalized site. The example method 161 further comprises, at step 164, subjecting the PKD cyst to a flow of a fluid having a volume, a solute concentration, and a flow rate. The example method 161 further comprises, at step 165, evaluating an effect of the flow of the fluid on the PKD cyst.

The human kidney organoid, and the iPS cell from which the human kidney organoid is derived, includes a genetic modification that is associated with PKD. While any genetic modification can be implemented to introduce a PKD genotype and phenotype, in particular embodiments, a PKD1 and/or a PKD2 gene is genetically deactivated to produce a PKD genotype and phenotype. The PKD1 and/or PKD2 gene can be genetically deactivated with any method in the art to produce any deactivated form of the genes; however, in example embodiments, the gene is truncated with a CRISPR/Cas9 gene editing technique. Accordingly, in embodiments, the method further comprises creating a PKD genotype in a human stem cell, and differentiating the human stem cell into the GM human kidney organoid. Genetic engineering techniques, such as any suitable CRISPR/Cas9 technique, can be used to knock down, knock out, or otherwise deactivate one or more genes to produce the PKD genotype, as described in more detail elsewhere herein.

In embodiments of the method for characterizing PKD in vitro, the method further comprises, e.g., before at least some steps of the method of FIG. 16, genetically deactivating a PKD2 gene in the human stem cell and/or the GM human kidney organoid to produce a PKD2 negative human stem cell and/or a PKD2 negative GM human kidney organoid. Alternatively, or in addition, the method can include genetically deactivating a PKD1 gene in the human stem cell and/or the GM human kidney organoid to produce a PKD1 negative human stem cell and/or a PKD1 negative GM human kidney organoid. Mutations to either or both of PKD1 and PKD2 can be used to produce induced pluripotent stem (iPS) cells that have the potential to form human kidney organoids with an inclination to form PKD cysts. The organoids can be produced through differentiation according to organoid culture techniques and can be induced to form PKD cysts according to methods of the disclosure.

In embodiments, methods for determining a mechanism of PKD cyst formation, expansion, and/or contraction in response to a property of the fluid are provided or implemented. The mechanism of PKD cyst formation can include a biomolecular mechanism, a macromolecular mechanism, a molecular mechanism, or another mechanism involved with PKD cyst formation, expansion, and/or contraction. The mechanism can relate to how PKD progresses and/or how PKD responds to a treatment or therapy.

In embodiments, the method comprises determining whether the PKD cyst absorbs glucose, determining whether glucose absorption increases PKD cyst formation, and/or determining a polarization of the PKD cyst. The polarization of the PKD cyst may be inverted, for example, as described in more detail herein. Previous iterations of culture systems and techniques did not enable these types of determinations, but the fluid-based systems of the disclosure enable these and other types of determinations and provide an improvement over other techniques such as monolayer culture techniques.

In embodiments, the method further comprises controlling a volume, a solute concentration, and/or a flow rate of the fluid as the property of the fluid, and determining the response of the PKD cyst to the volume, the solute concentration, and/or the flow rate of the fluid. These or other properties of the fluid can be controlled to mimic or model the kidney microenvironment and determine how the PKD cyst responds to the changing model system microenvironment. In this manner, the investigator or person of skill in the art can better utilize and rely on the kits and systems of the disclosure for their modeling of the PKD kidney microenvironment as a PKD disease model system.

In embodiments, a flow of the fluid corresponds with, or causes, formation and/or expansion of the PKD cyst. Since fluid flow increases formation of the PKD cyst, fluid flow can be used as part of a method for generating the PKD cyst for further study, as described in more detail below. In embodiments, the inlet is fluidly connected to the outlet and the fluid flows from the inlet through the channel toward the outlet according to the flow rate and flows over the PKD cyst of the GM human kidney organoid. Since the system closely models fluidic flow in the kidney microenvironment, alterations to the model system can be used to model changes that might occur in the body, for example, in therapy and/or non-therapy scenarios. In this manner, the investigator or person having skill in the art can rely on the systems and kits for accurate modeling of the human kidney microenvironment.

In embodiments, the method for characterizing PKD in vitro further comprises contacting the PKD cyst with a glucose transport inhibitor to determine an effect of glucose transport inhibition on PKD cyst formation, expansion, and/or contraction. It is described herein that the presence of the glucose transport inhibitor in the fluid impacts PKD cyst formation, for example. In embodiments, the glucose transport inhibitor comprises phloretin, phloridzin, and/or dapagliflozin. Inclusion of one or more of these or other glucose transport inhibitors can be implemented to determine whether glucose transport is associated with PKD cyst formation.

In another aspect of the disclosure, a method for formation of a PKD cyst of a GM human kidney organoid for characterizing PKD in vitro comprises culturing the GM human kidney organoid within a flow device and contacting the PKD cyst with a flow of the fluid. The flow of the fluid increases formation of PKD cysts within the model system. The flow device includes a channel with a functionalized site configured for cell culture and an inlet and an outlet configured for flow of a fluid at the functionalized site. In embodiments, the human kidney organoid has a genetic modification that is associated with PKD. Any genetic modification can be implemented to cause a PKD phenotype, having PKD cysts, when the GM human kidney organoid is cultured. The system and method can reliably and reproducibly form PKD cysts for further study of the PKD disease state.

In embodiments of the method for formation of a PKD cyst, the method further comprises contacting the PKD cyst with glucose to increase formation of the PKD cyst. Increased formation of the PKD cyst in the presence of glucose can be implemented in a method to produce PKD cysts for studying a mechanism of the PKD disease state, for example. In embodiments, the characterization of the PKD disease state comprises determining a mechanism of PKD cyst formation, expansion, and/or contraction in response to the fluid. In instances where the PKD cyst is formed, a person of skill in the art can determine the mechanism responsible by varying the properties of the fluid and determining characteristics of formation of the PKD cyst as a result of the properties of the fluid.

In embodiments of the method for formation of a PKD cyst, the method further comprises controlling a volume, a solute concentration, and/or a flow rate of the fluid as the property of the fluid; and determining the response of the PKD cyst to the volume, the solute concentration, and/or the flow rate of the fluid. These or other properties of the fluid can be controlled to mimic the kidney microenvironment and control how the PKD cyst responds to the changing model system microenvironment. In embodiments, the flow of the fluid corresponds with formation of the PKD cyst. In such instances, the PKD cyst is more readily formed as a result of the flow of the fluid.

In embodiments, the inlet is fluidly connected to the outlet and the fluid flows from the inlet through the channel toward the outlet according to the flow rate and flows over the PKD cyst of the GM human kidney organoid. Since the system closely models fluidic flow in the kidney microenvironment, alterations to formation of the PKD cyst in the model system can be used to model changes that might occur in the body, for example, in therapy and/or non-therapy scenarios, that impact formation of a PKD cyst in the human body.

### EXAMPLES

### Example 1: Absorptive Mechanism of Polycystic Kidney Disease

This example is directed to compositions, systems, and methods for studying disease mechanisms in kidneys and other organs. In particular, it relates to therapeutics, diagnostics, and regenerative applications.

### Flow Induces Cyst Swelling in PKD Organoids

Prior to introducing flow, the specificity and timing of the PKD phenotype was confirmed in static cultures. PKD1^{-/-} or PKD2^{-/-} hPSC were differentiated side-by-side with isogenic controls under static, adherent culture conditions to form kidney organoids. On day 18 of differentiation, prior to cyst formation, organoids were carefully detached from the underlying substratum and transferred to suspension cultures in low-attachment plates. Under these conditions, the majority of PKD1^{-/-} or PKD2^{-/-} organoids formed cysts within 1-2 weeks, whereas isogenic control organoids rarely formed cysts (FIG. TA). In repeated trials, the difference between PKD organoids and isogenic controls was quantifiable and highly significant (FIG. 1A). Thus, PKD organoid formed cysts in a genotype-specific manner, strongly suggesting that this phenotype was specific to the disease state. This differs from other types of three-dimensional cultures of epithelial cells, in which hollow ‘cysts’ (i.e., in actuality, spheroids) arise irrespective of PKD genotype and represent a default configuration of the epithelium rather than a disease-specific phenotype.

To understand how flow affects PKD in organoids, a microfluidic system was designed that allows for live imaging of kidney organoids during the early stages of cyst formation (FIG. 1B). Genetically modified hPSC were first differentiated into organoids under static, adherent culture conditions for 26 days, at which time point tubular structures had formed with small cysts in the PKD cultures. Organoids were then purified by microdissection and transferred into gas-permeable, tissue culture-treated polymer flow chambers (0.4 mm height×3.8 mm width), which were optically clear and large enough to comfortably accommodate organoids and cysts. The channels were pre-coated with a thin layer of Cornel® Matrigel® for functionalization, and organoids were allowed to attach to the Matrigel® overnight. PKD and isogenic control organoids were subjected to fluid flow with a wall shear stress of 0.2 dynes/cm², which approximates physiological shear stress within kidney tubules. In these devices, it was observed that cysts in PKD organoids increased in size rapidly under flow (change in area of ˜20,000 μm²/hr, or ˜160 m/hr in diameter), compared to non-cystic compartments within these organoids, or isogenic control organoids lacking PKD mutations, which did not swell appreciably (FIG. 1C and FIG. 1D).

### Diffusion can Partially Substitute for Flow

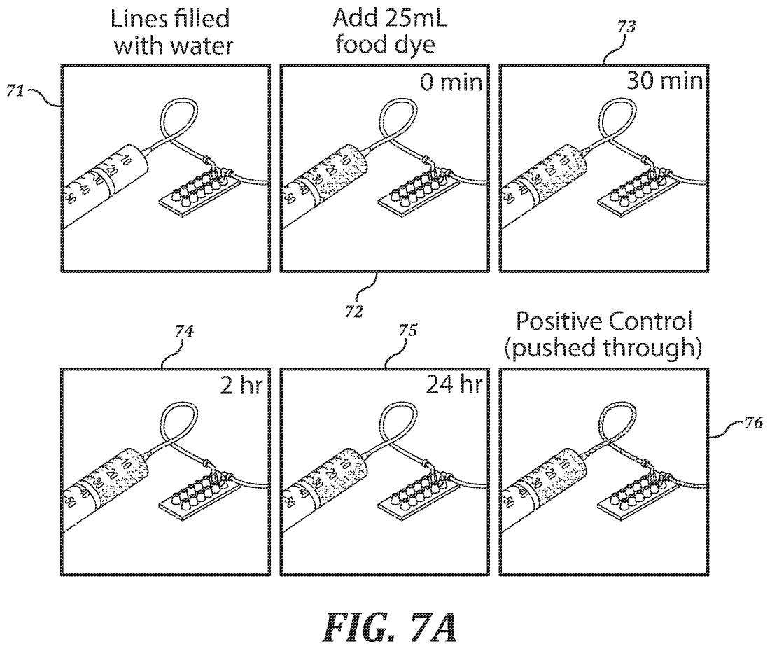

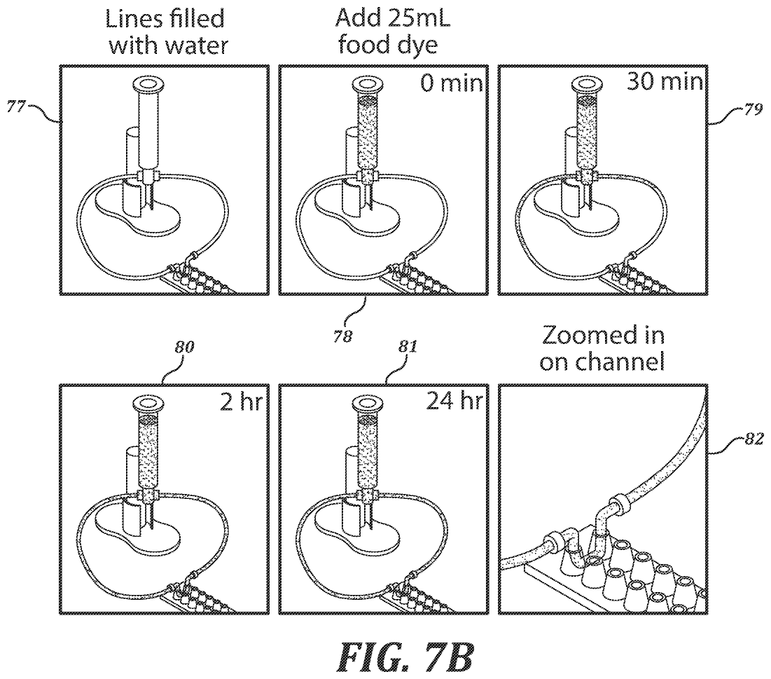

Having observed that cysts expand under microfluidic conditions, it was next sought to establish a corresponding static condition lacking flow as a negative control. Initially the same chambers and syringe pump were utilized in the absence of pump activation, which is a commonly used control format for microfluidic experiments. However, it was observed that food dye contained within the syringe failed to enter the microfluidic chamber under these conditions (see FIG. 7A, steps 71, 72, 73, 74, 75, 76). This indicated a lack of diffusion, which meant that organoids would be exposed only to the volume of media present within the channel of the microfluidic device (˜200 μL), which was much lower than the volume they would encounter under fluidic conditions (˜60 mL/6 hr). Such a static condition could not be readily compared to fluidic conditions to determine the effects of flow, since other parameters such as volume and total solute mass would also be very different.

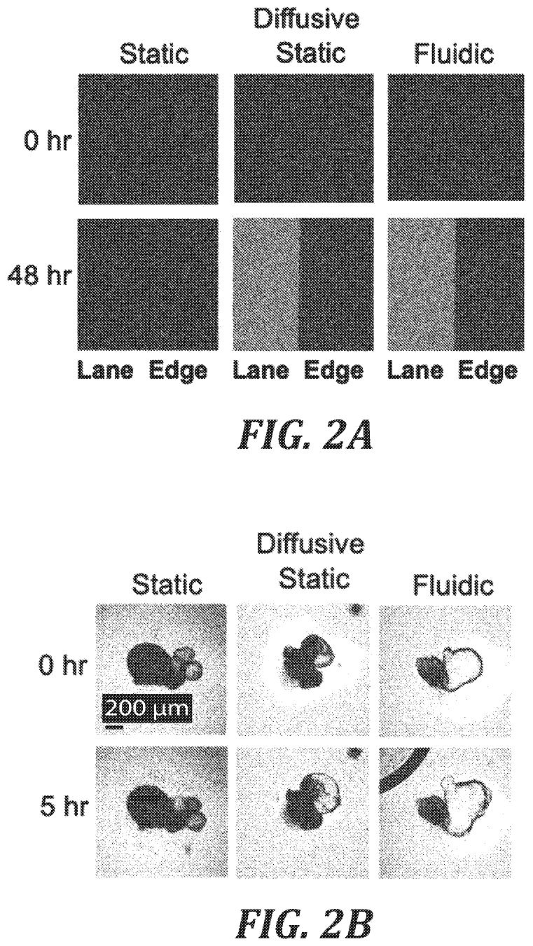

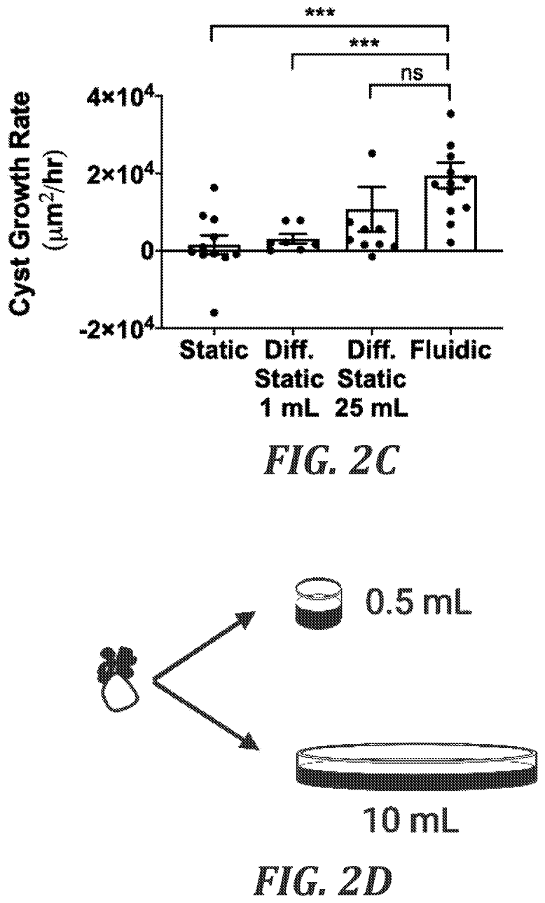

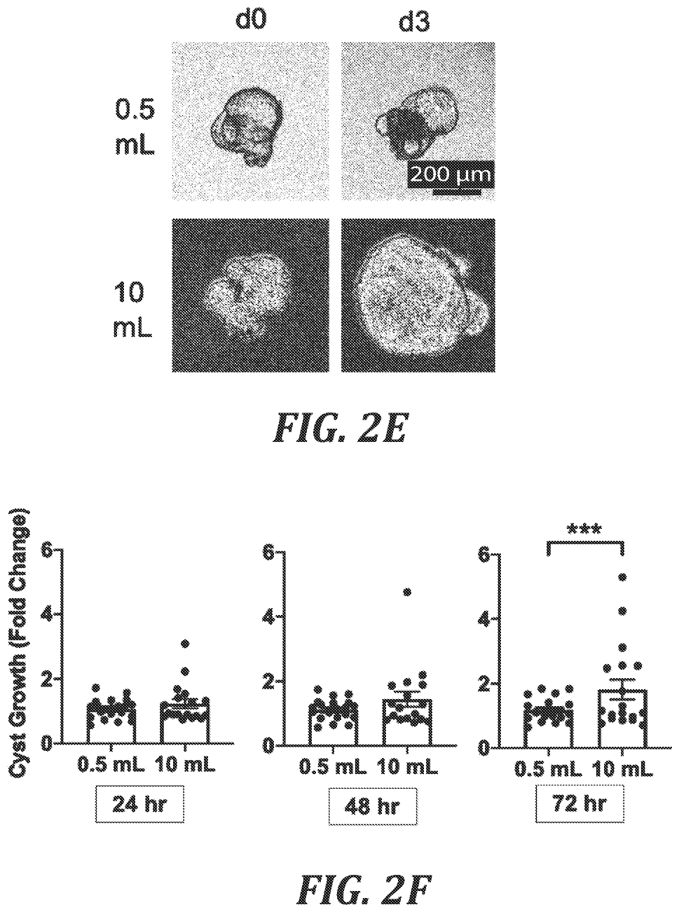

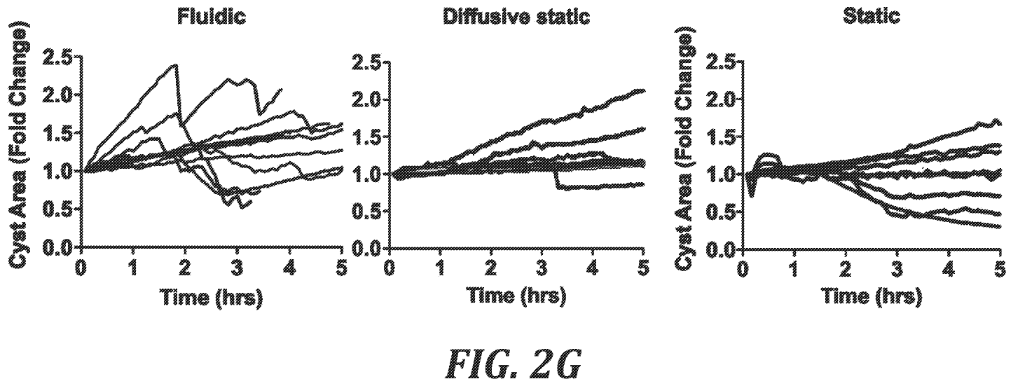

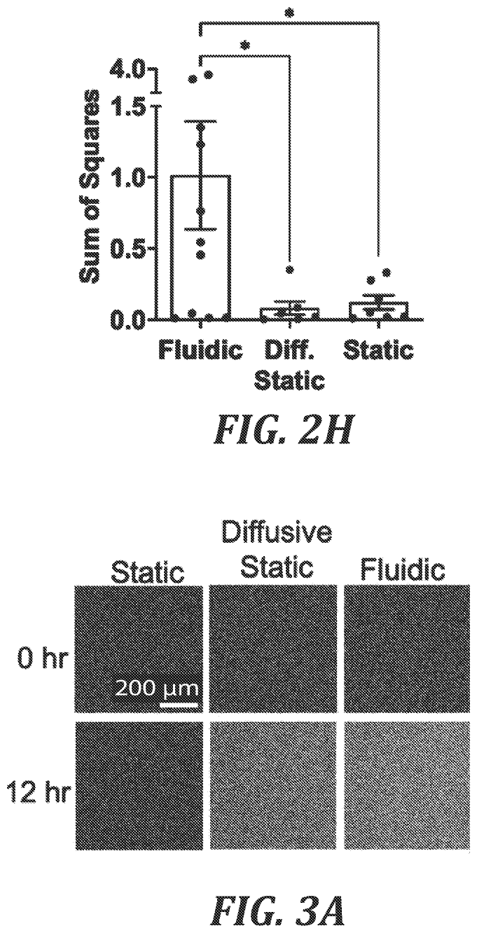

To control for the effects of flow more accurately, a diffusive static condition was designed that exposed organoids to an equivalent volume of culture media as in the flow condition. This included a reservoir of media (maximum volume of 25 mL) connected to the microfluidic chip by wider tubing to allow for efficient and uninhibited diffusion of small molecules into the microfluidic channel. In this static format, food dye diffused from the media reservoir into the channel after 2-3 hours (see FIG. 7B, steps 77, 78, 79, 80, 81, 82). Similarly, rhodamine-labeled dextran (10 kDa) diffused from the media reservoir into the channel and equilibrated with fluidic epifluorescence within 48 hours (FIG. 2A).

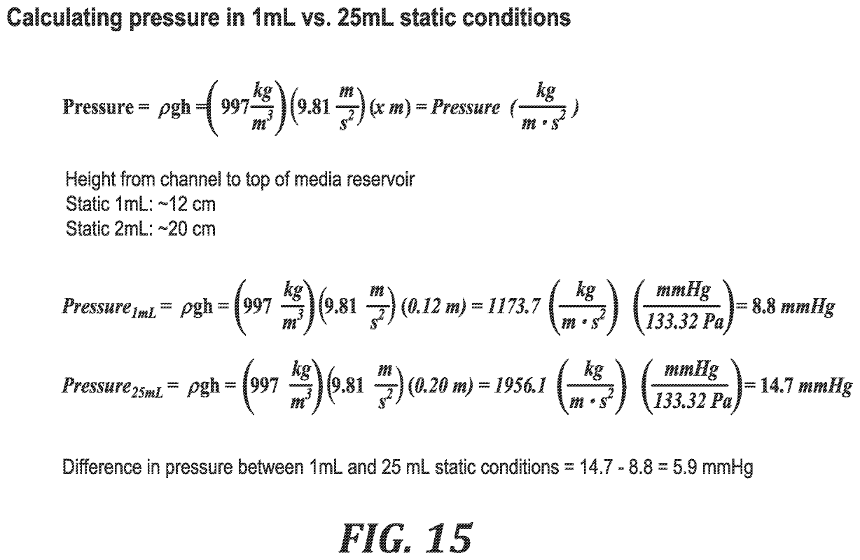

To further validate this ‘diffusive static’ condition, the volume of media in the reservoir was varied and cyst growth analyzed over a period of 12 hours. Cysts exposed to a reservoir containing 1 mL of media expanded at a rate of ˜3,000 μm²/hr, whereas a reservoir containing 25 mL increased expansion to ˜10,000 m²/hr, approximately half the rate observed in the fluidic condition (FIG. 2B and FIG. 2C). Using the equation Pressure=ρgh, the hydrostatic pressure on organoids with 1 mL and 25 mL media reservoirs was calculated to be 1174 Pa and 1956 Pa, respectively. As this represented a substantial pressure difference of 5.9 mmHg (FIG. 15), experiments were conducted to distinguish between the effects of pressure versus volume on cyst growth. Cystic organoids were suspended in either 500 μL or 10 mL, with a constant fluid column height of 1 cm (FIG. 2D). Cysts exposed to 10 mL of media grew significantly more than those exposed to 500 μL of media (FIG. 2E and FIG. 2F). Thus, media volume was identified as a major determinant of expansion that could partially substitute for flow in this system.

Not all aspects of the fluidic condition were replicated by the diffusive static condition. Time-lapse microscopy under continuous flow revealed that PKD cysts exhibited fluctuating growth profiles, expanding and constricting (deflating) in cyclical, “breath-like” movements. Constrictions occurred rapidly when the cysts appeared to be fully inflated, suggesting that they resulted from rupture of the epithelium, for instance in response to expansive fluid force (FIG. 2G). Growth and constriction events occurred within hours after the initiation of flow, indicating a rapid physical mechanism rather than a slower one based on cell proliferation. This oscillatory behavior was unique to the fluidic condition and was not observed in either the diffusive static or non-diffusive static conditions, nor in non-cystic controls (FIG. 2G). Using the sum of squares method, it was found that cyst dynamics (variance in size within an individual structure over time) were much greater in the fluidic condition, compared to either of the static conditions (FIG. 2H). As solute exposure was likely to occur much more rapidly in the fluidic condition, solute uptake was examined under these conditions.

### Cysts Absorb Glucose During Flow-Mediated Expansion

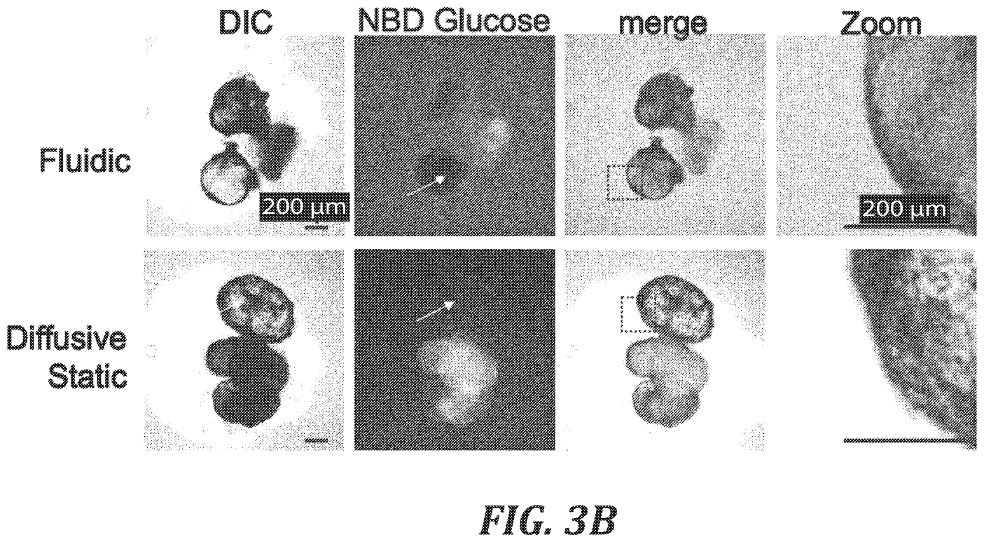

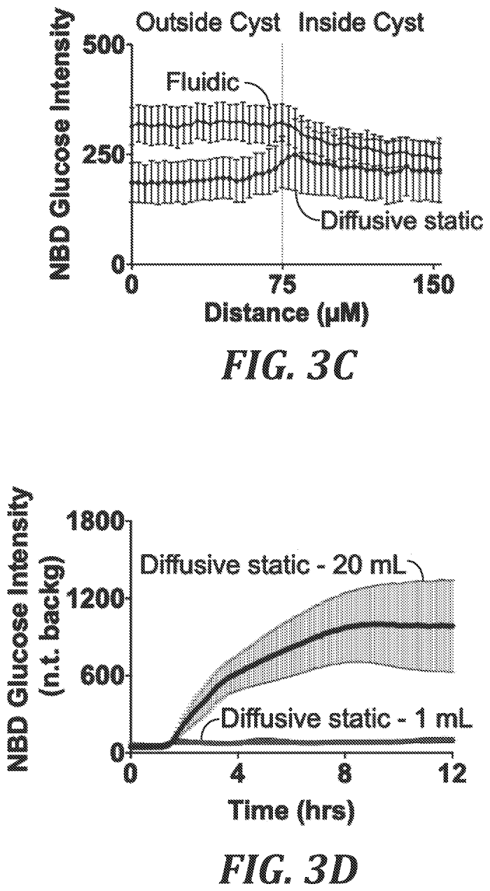

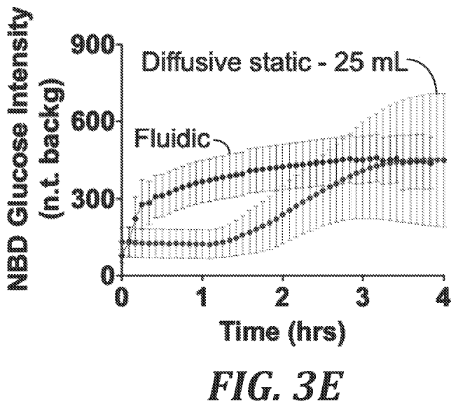

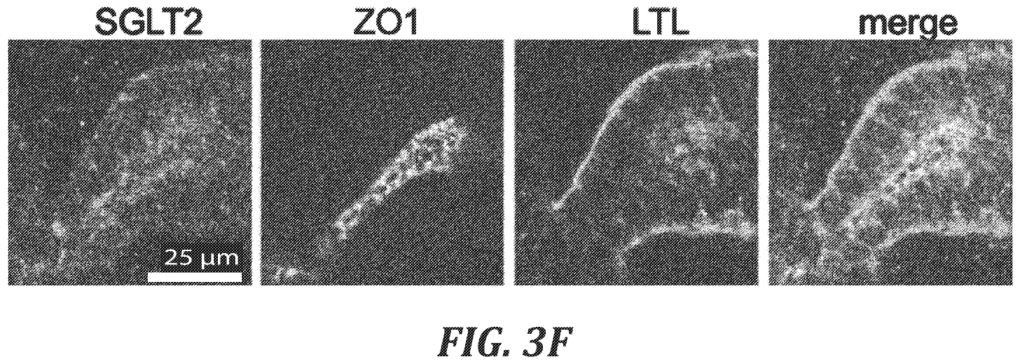

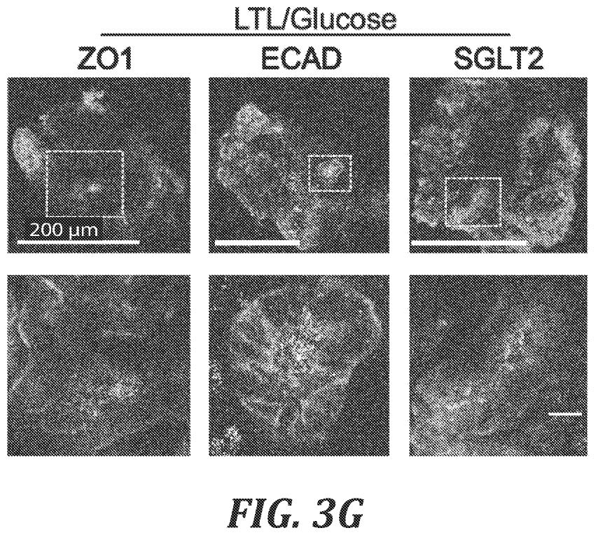

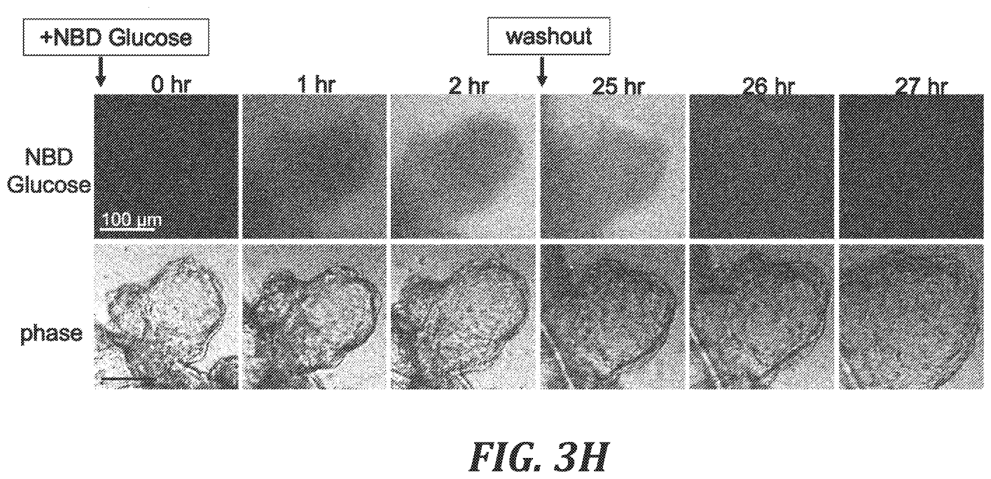

Glucose is an abundant renal solute and transport cargo, which might explain the effects of media exposure on cyst expansion, but whether kidney organoids absorb glucose is unknown. Glucose transport in cysts and organoids was therefore studied using a fluorescent glucose analog, 2-(N-(7-Nitrobenz-2-oxa-1,3-diazol-4-yl)Amino)-2-Deoxyglucose (NBD-Glucose). The low height of the channels in the flow devices enabled continuous time lapse imaging of fluorescent molecules without high background fluorescence. Glucose was observed to infiltrate into the devices under both diffusive static as well as fluidic conditions. Epifluorescence of NBD-Glucose gradually increased and plateaued at similar levels after 12 hours in both the diffusive static condition and the fluidic condition, but did not accumulate detectably within the channels in the non-diffusive static condition (FIG. 3A).

When this assay was performed in channels seeded with organoids, PKD cysts absorbed glucose under fluidic and diffusive static conditions (FIG. 3B). Line scan analysis of these images showed that there was no significant difference in absorption between the fluidic and diffusive static conditions (FIG. 3C). Analysis of glucose absorption in organoid tubules over time confirmed that the volume of media in the static condition was a factor in nutrient absorption (FIG. 3D). Glucose absorption in organoids over time under the diffusive static condition followed an S-shaped absorption curve, whereas glucose levels in the fluidic condition increased rapidly and then plateaued, approximating an exponential curve, but both conditions plateaued at approximately the same maximal level of glucose absorption (FIG. 3E). These studies suggested that flow has no additional effect on glucose absorption in organoids when compared to a static control presenting equivalent total glucose exposure.

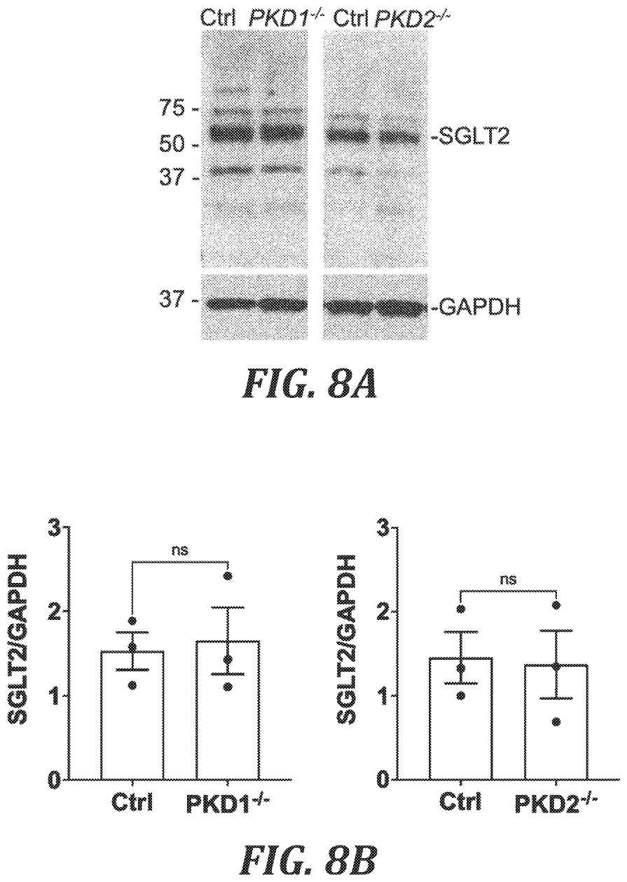

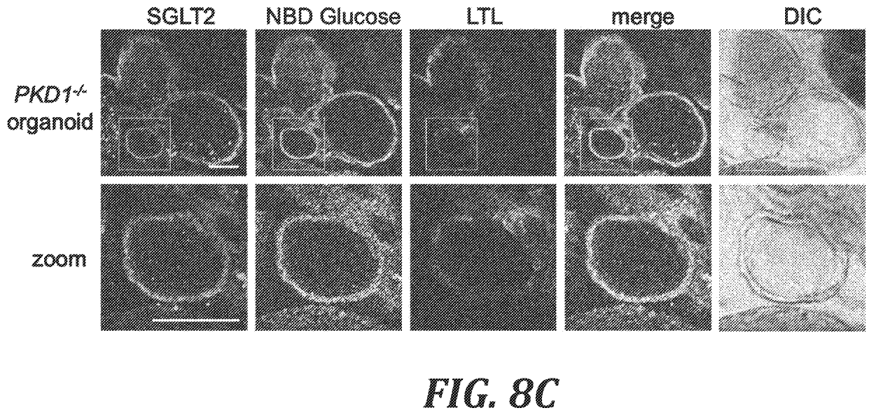

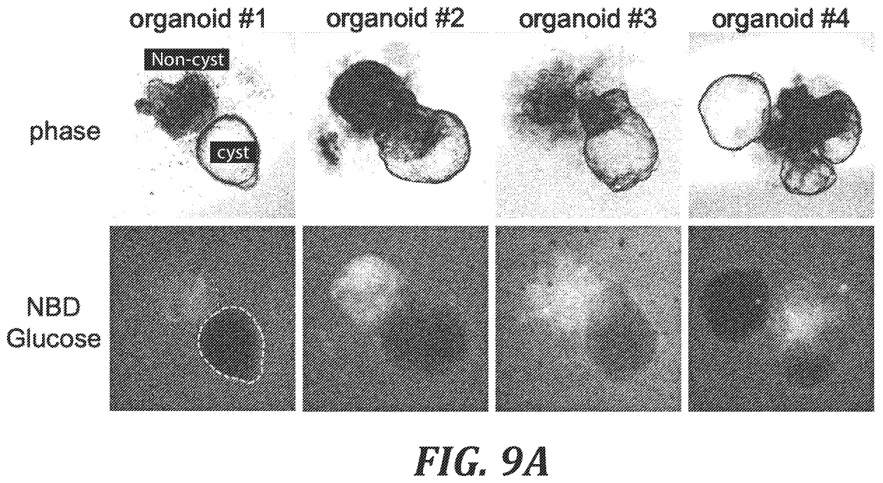

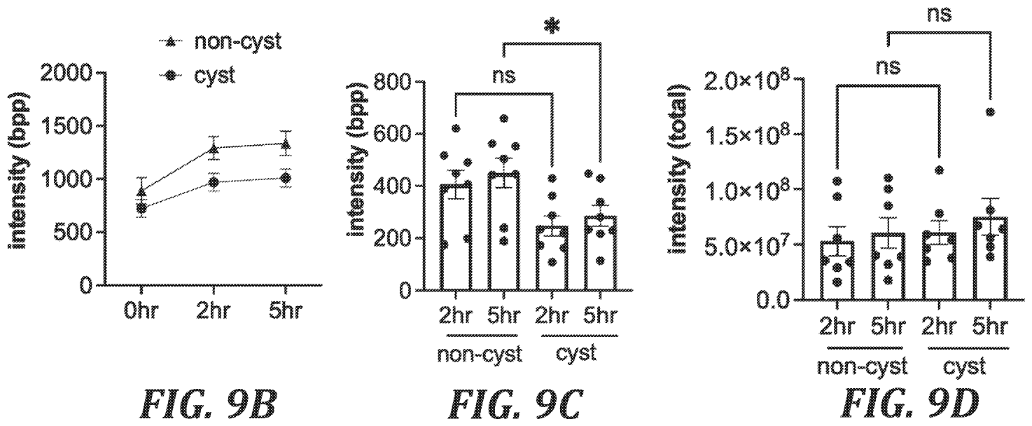

Glucose absorption was a general property of kidney organoids. In non-cystic structures, sodium-glucose transporter-2 (SGLT2) was expressed in organoid tubules and enriched at the apical surface, delineated by the tight junction marker ZO-1 (FIG. 3F). Immunofluorescence confirmed that NBD glucose was absorbed into and accumulated inside organoid proximal and distal tubules (FIG. 3G). Immunoblot analysis indicated similar levels of SGLT2 in control and PKD organoid cultures (FIG. 8A, FIG. 8B). Cyst-lining epithelia expressed SGLT2, and accumulated glucose both intracellularly as well as inside their lumens (FIG. 8C). Intracellular glucose levels were generally higher than extracellular levels, consistent with the tendency of NBD glucose to accumulate inside cells (FIG. 9A, FIG. 9B, FIG. 9C). Although cysts were much less cell-dense than attached non-cystic compartments, cystic and non-cystic compartments accumulated similar total levels of glucose, owing to the larger size of the cysts (FIG. 9D). When PKD organoids loaded with NBD glucose were switched into media containing only unlabeled glucose (washout), NBD glucose disappeared rapidly from these structures (FIG. 3H). Thus, organoids continuously accumulated and released glucose in a dynamic fashion.

### Inhibition of Glucose Transport Blocks Cyst Growth

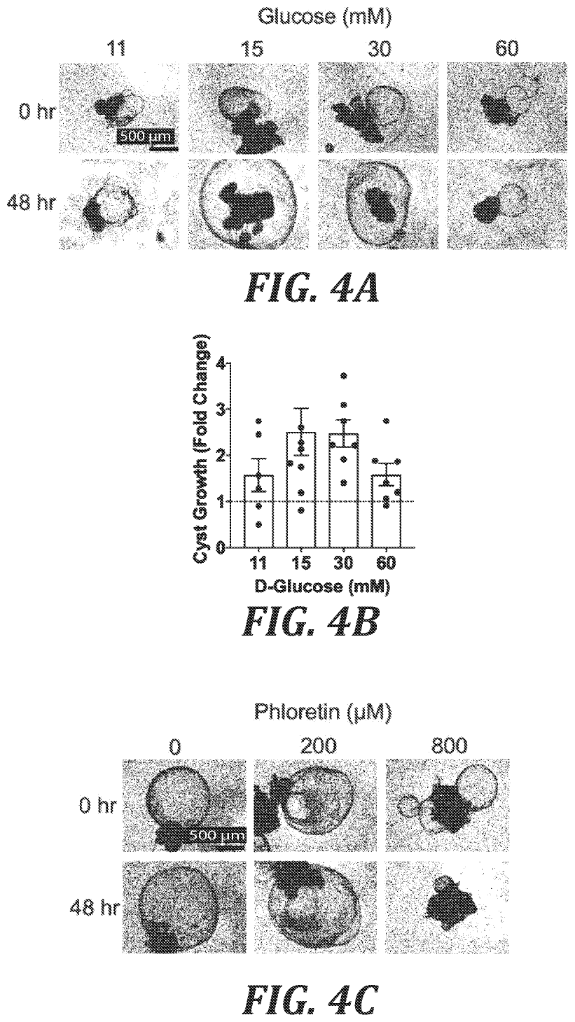

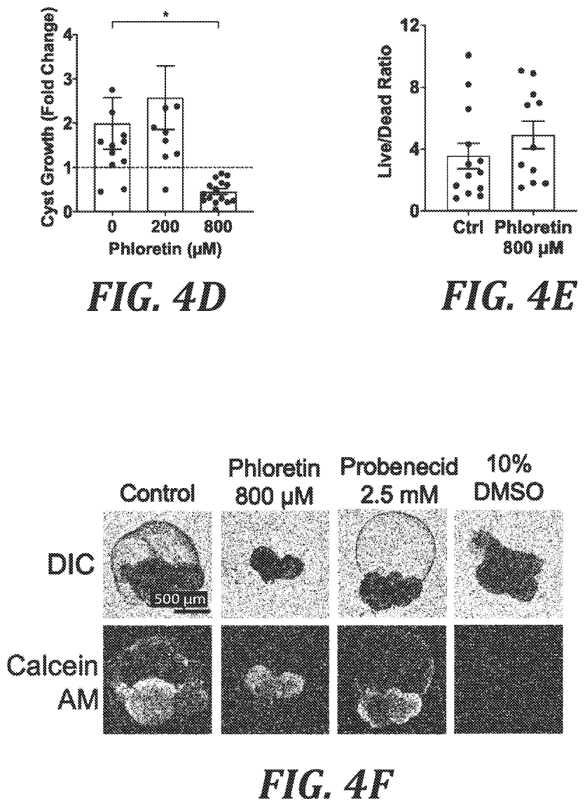

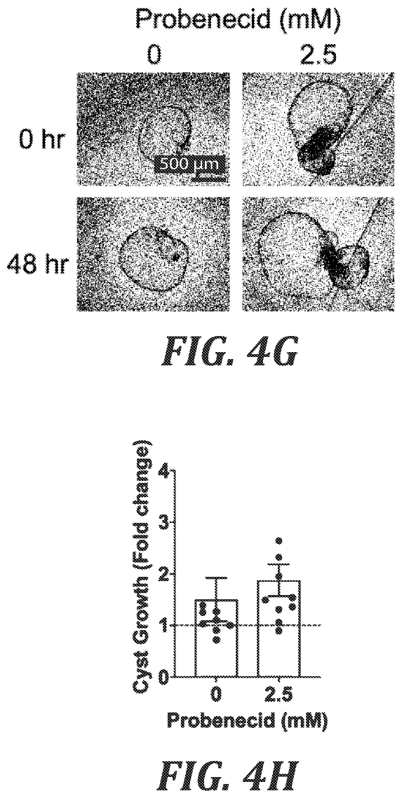

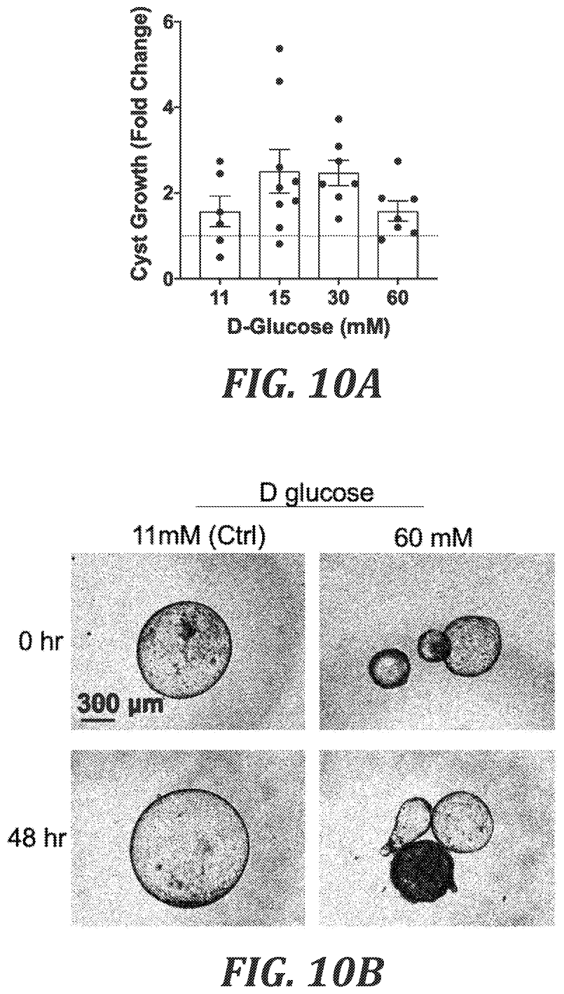

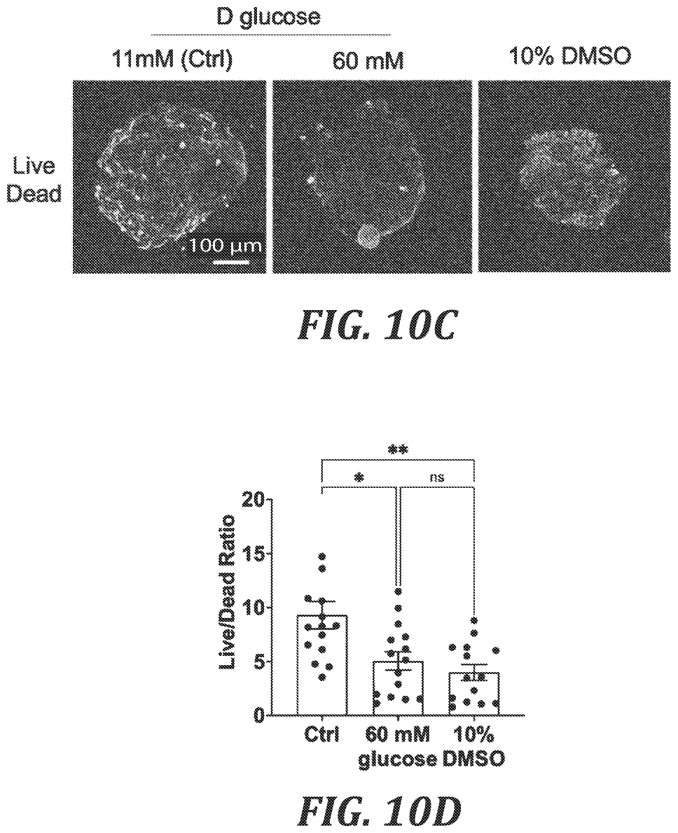

In animal models, inhibitors of glucose transport are suggested to have both positive and negative effects in PKD. To test functionally whether cyst growth is linked to glucose transport in human organoids, cyst expansion was quantified in increasing concentrations of D-glucose under static conditions (96-well plate). Growth was maximal at 15-30 mM glucose, causing ˜50% increase in cyst expansion, relative to lower or higher concentrations (FIG. 4A, FIG. 4B, FIG. 10A). Live/dead analysis of cysts treated with 60 mM glucose detected cytotoxicity, explaining the reduction in cyst growth at this higher concentration (FIG. 10B, FIG. 10C, FIG. 10D).

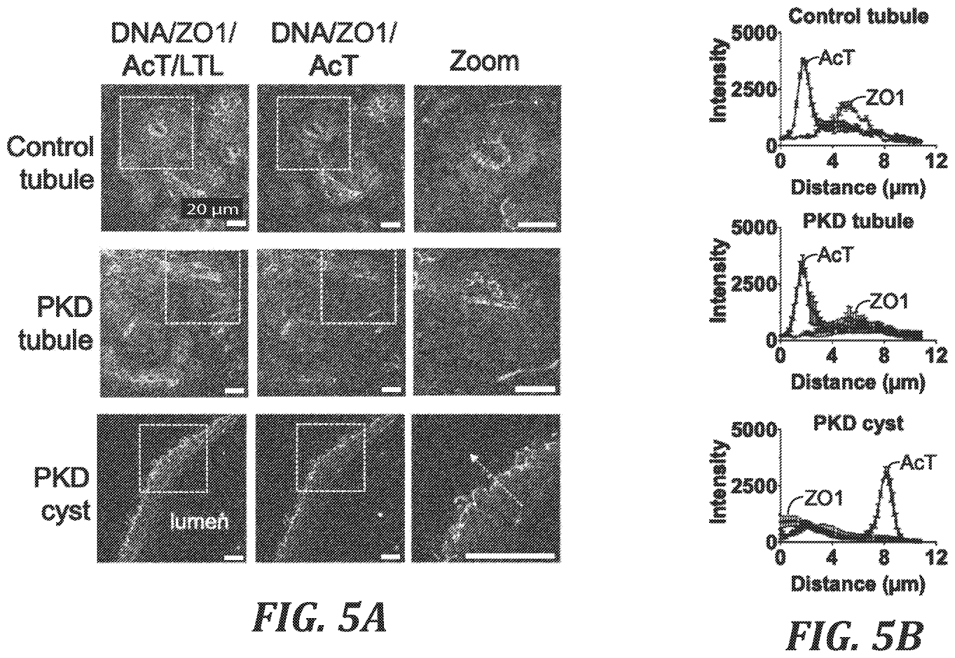

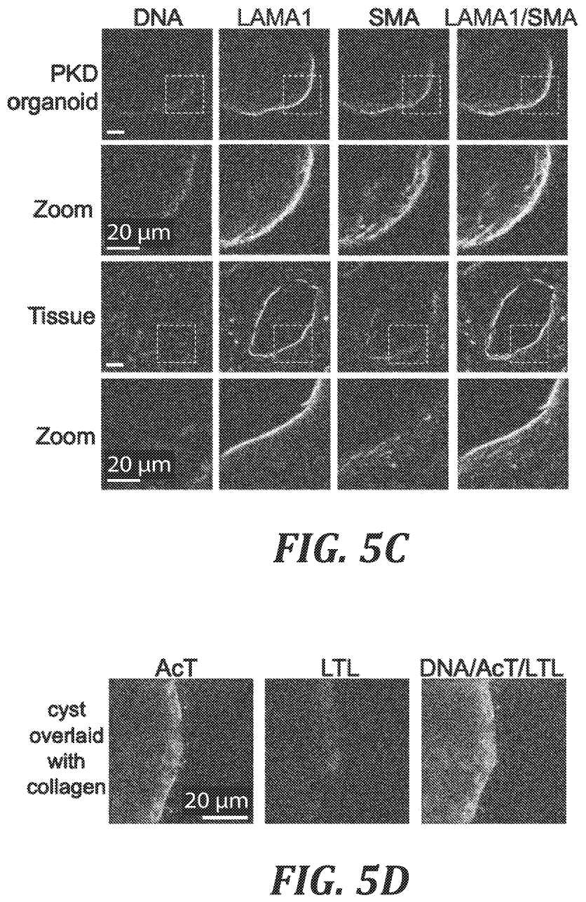

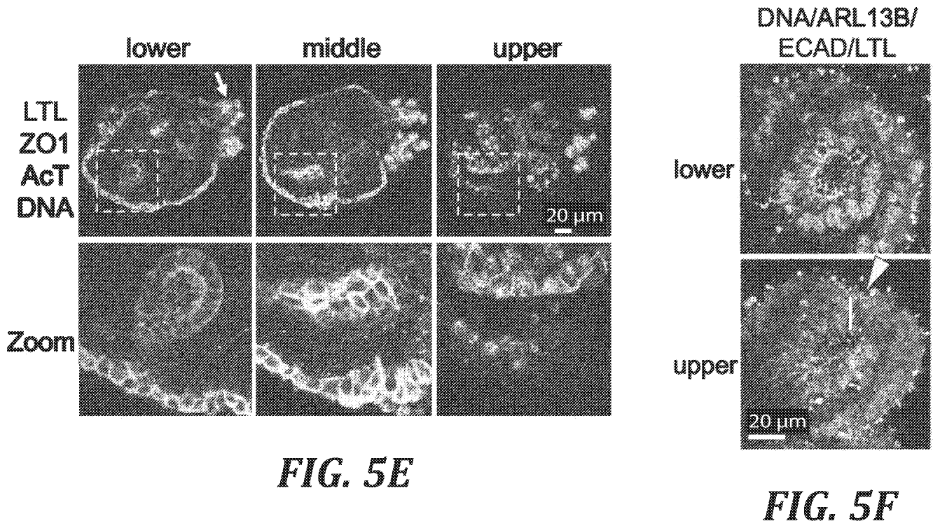

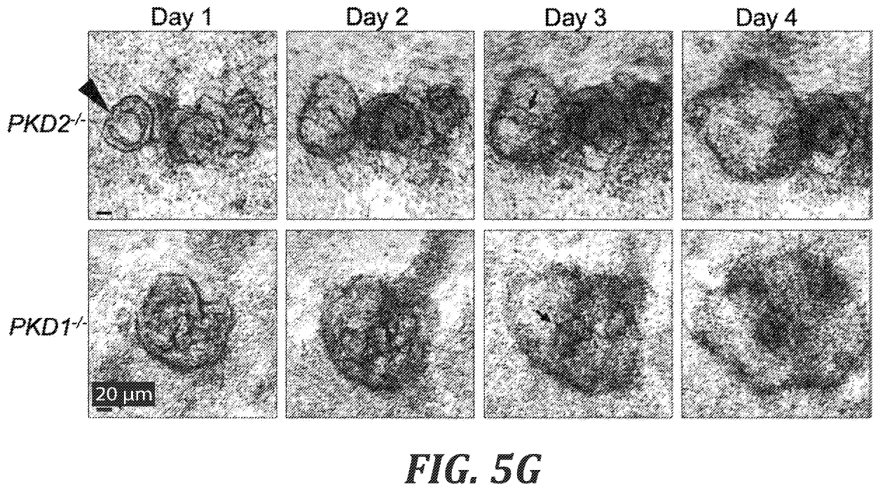

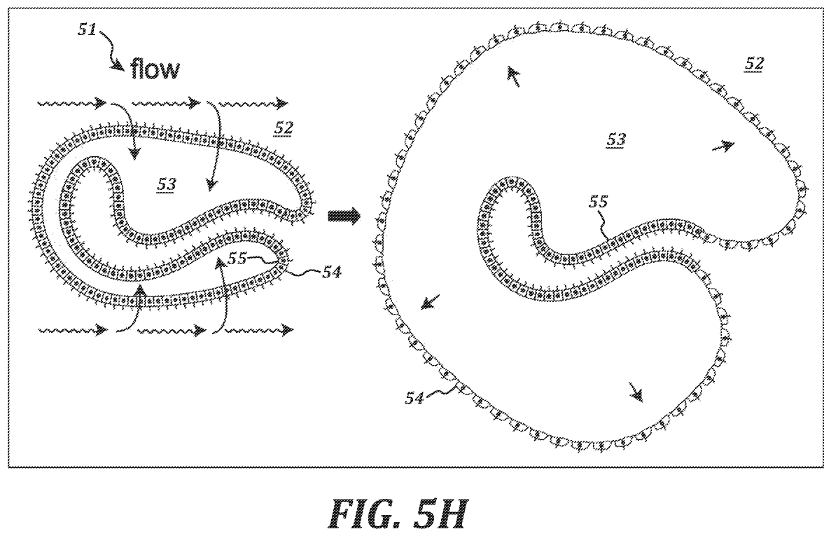

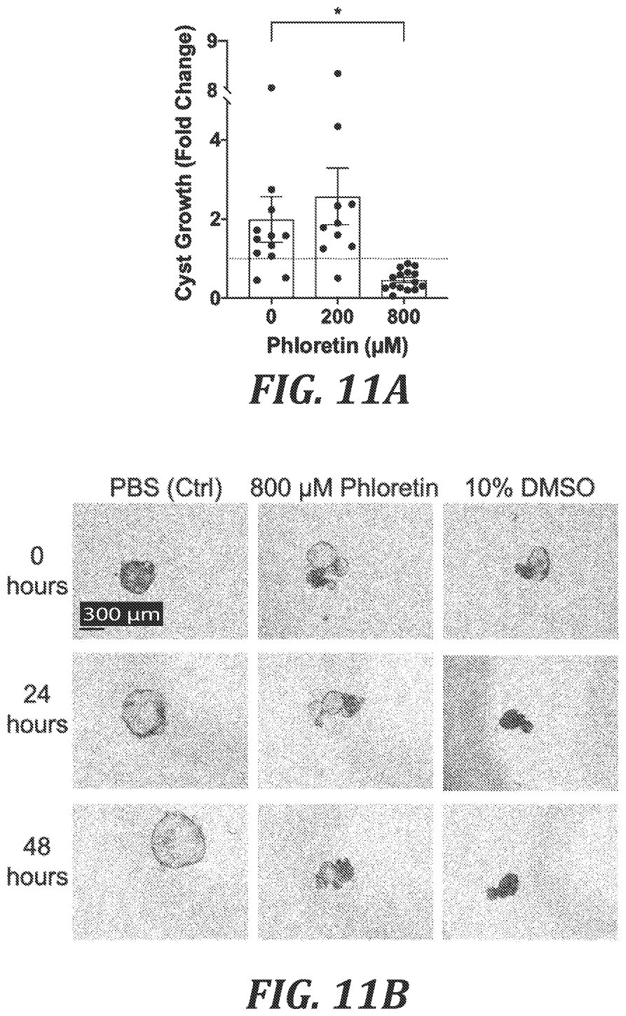

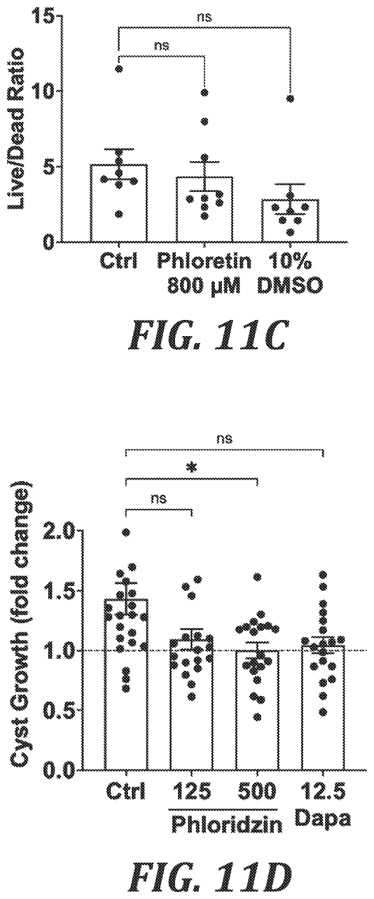

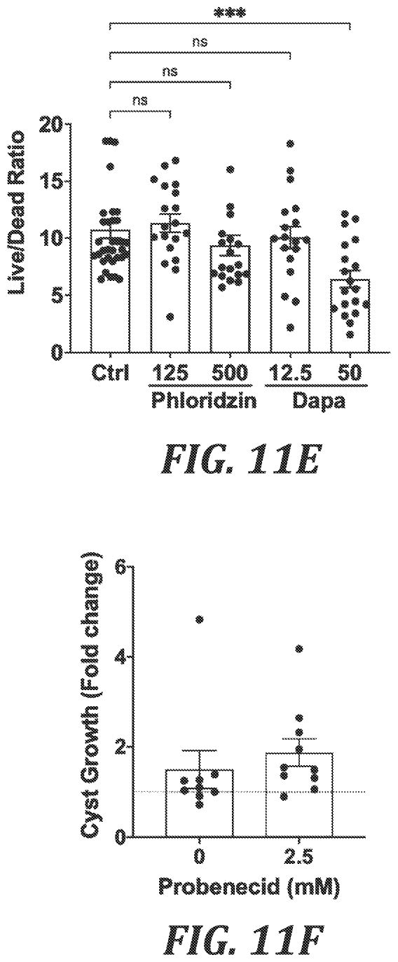

The preceding findings, together with the rapid turnover of glucose in organoids described above, suggested that inhibition of glucose import might enable export mechanisms to dominate, resulting in blockade or even reversal of cyst growth due to osmotic effects. To test this hypothesis, the effects of pharmacological transport inhibitors on cysts were examined in static conditions. Phloretin, an inhibitor of sodium/glucose cotransporters 1 and 2 (SGLT1 and SGLT2), was tested in 15 mM glucose, and found to decrease cyst size by 77% at a concentration of 800 μM (FIG. 4C, FIG. 4D, FIG. 11A). Live-dead staining at 24 and 48 hours of phloretin treatment revealed no significant toxicity (FIG. 4D, FIG. 4E, FIG. 5B, FIG. 5C). Treatment with either phloridzin, a non-selective inhibitor of both SGLT1 and SGLT2, or with dapagliflozin, a specific inhibitor of SGLT2, reduced cyst growth to baseline at non-toxic doses, further supporting the hypothesis (FIG. 11D, FIG. 11E). Without wishing to be bound by any particular theory, net shrinkage of cysts was not observed with phloridzin or dapagliflozin, suggesting either decreased potency of these compounds relative to phloretin, or an off-target effect of phloretin beyond glucose transport that further reduces cyst size. In contrast to SGLT inhibitors, probenecid, an inhibitor of the OAT1 transporter on the basolateral membrane, had no effect on cyst growth compared to controls at non-toxic doses (FIG. 4F, FIG. 4G, FIG. 4H, FIG. 5F). Overall, these findings supported the hypothesis that pharmacological inhibitors specific for sodium/glucose cotransporters (SGLTs) block cyst expansion in the PKD organoid model, consistent with a role for glucose accumulation in cyst expansion.

### Organoid Cysts Polarize Outwards

Some previous studies have suggested that cyst expansion may be due to increased secretory (basolateral-to-apical) solute transport. However, glucose transport in the proximal tubule is predominantly reabsorptive (apical-to-basolateral) rather than secretory. To better understand the directionality of transport within organoids, the apicobasal polarity of tubules and cysts was determined using antibodies against tight junctions and cilia. In both PKD and control organoids, the ciliated surface of these tubules faced inwards (FIG. 5A). Interestingly, however, PKD cysts were polarized with the apical ciliated surface facing outwards towards the media and exposed to flow (FIG. 5A). Thus, the external cyst surface resembled the apical surface of a tubule in this system. Line scan analysis confirmed this inverted polarization, with primary cilia and tight junction intensity profiles reversed in organoids vs. cysts (FIG. 5B).

Close examination of PKD organoid cysts revealed that a subpopulation of these contained a layer of cells expressing alpha smooth muscle actin immediately beneath the cyst-lining epithelium, which formed a laminin-rich basement membrane (FIG. 5C). In contrast, in human kidney tissue the basement membrane and myofibroblast-like cells surrounded cysts externally (FIG. 5C). Thus, apical cell polarity aligned opposite the basement membrane in both systems. It is reported that simple spheroids of Madine-Darby Canine Kidney cells in suspension culture polarize outwards, but can reverse apicobasal polarity from outwards to inwards when embedded in collagen. When the PKD cysts in organoids were overlaid with collagen, however, cyst polarity remained inverted and did not repolarize with the ciliated surface facing away from the extracellular matrix. Without wishing to be bound by any particular theory, this result could indicate that organoid cyst polarity was deeply entrenched and governed by more dominant, internal cues (FIG. 5D).

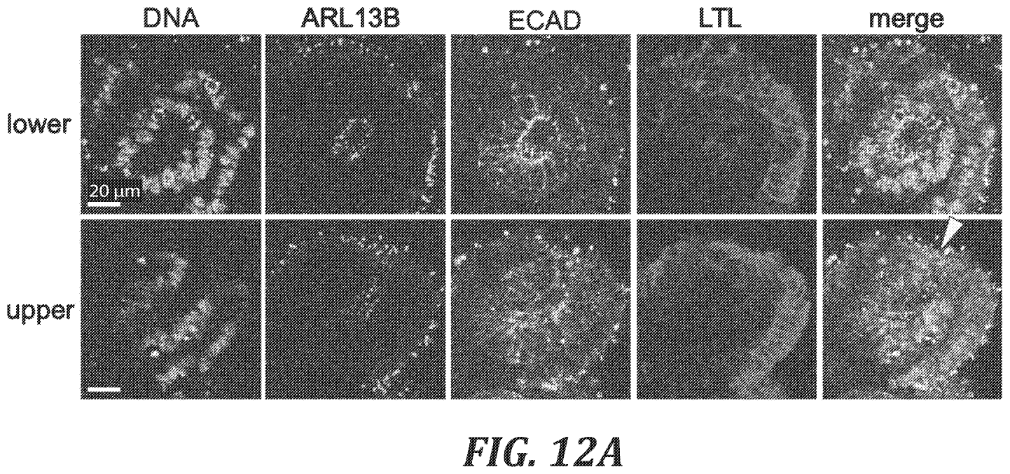

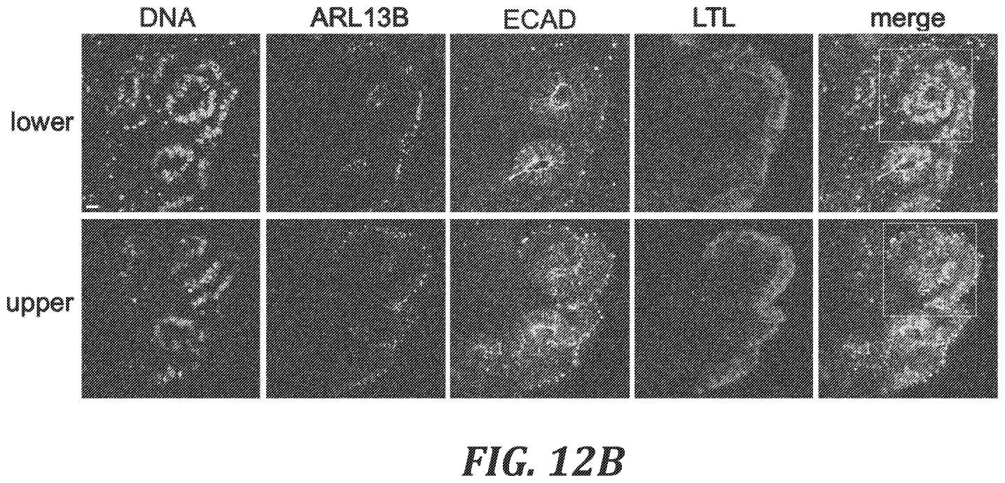

The observation that cysts polarized outwards seemed counter-intuitive, as tubule structures in human kidney organoids typically polarize inwards, with tight junctions and apical markers abutting one another from diametrically opposed epithelia (as shown in FIG. 5A). To resolve this apparent conundrum, PKD organoids were closely examined in three-dimensional confocal image z-stacks. Lotus tetragonolobus lectin (LTL), which is expressed more strongly in tubules than in cysts, was used to label the epithelium, while primary cilia and ZO-1 were used to indicate cell polarity. These experiments revealed that young cysts comprised epithelial spheroid structures (predominantly LTL⁺) with underlying tubular infolds, which faced inwards (FIG. 5E). Organoids were further examined without cysts (controls) in confocal microscopy z-stacks. It was noted that epithelium lining the periphery of these organoids faced outwards, whereas ‘tubules’ internal to organoids were invaginations of this peripheral epithelium (FIG. 5F, FIG. 12A, FIG. 12B). The innermost regions of these invaginated tubules were enriched for ECAD, a marker of distal tubule, whereas the external peripheral epithelia were enriched for LTL, a marker of proximal tubule (FIG. 5F, FIG. 12A). Thus, organoids constituted a continuous, proximal-to-distal epithelium, with the apical surface polarized outwards on the peripheral (more proximal) epithelium and inwards in the internal (more distal) epithelium of the structure.

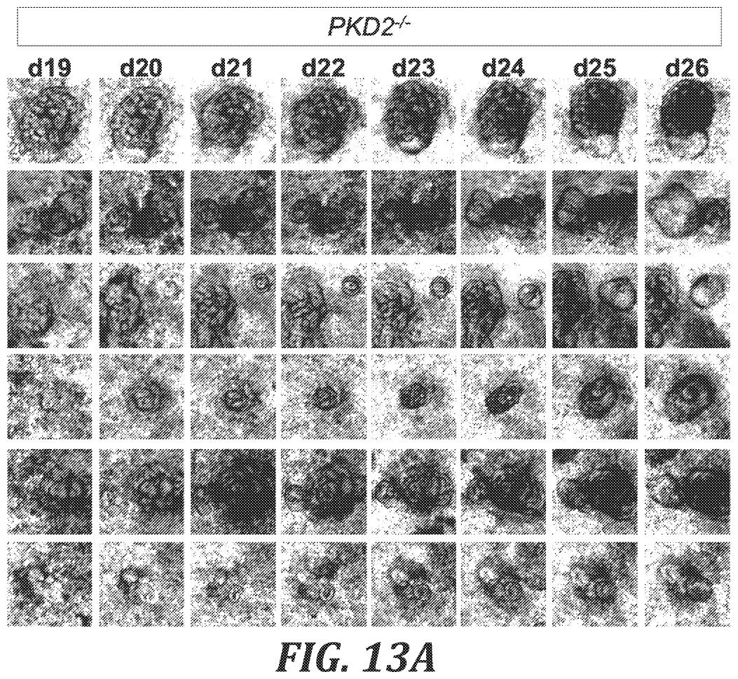

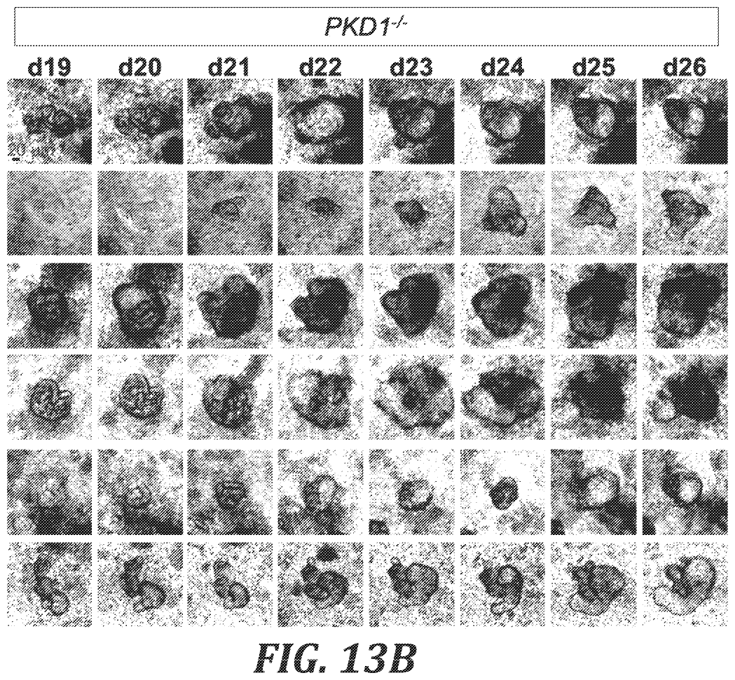

To observe the process of cyst formation in real time, time-lapse images of young PKD organoids undergoing cystogenesis over eight days in culture were collected. Consistently, cysts formed at the periphery of the organoids (FIG. 5G, FIG. 13A, FIG. 13B). During the early stages of cystogenesis, tubular structures remained visible inside the cysts as they expanded (FIG. 5G, FIG. 13A, FIG. 13B). Without wishing to be bound by any particular theory, the time-lapse imaging supported the idea that cysts formed from the peripheral epithelium of the organoids that faced outwards towards the media, rather than from the internal tubular invaginations, which tended to stay anchored (FIG. 5H), and this was consistent with an absorptive mechanism mediated by the peripheral epithelium.

### Absorptive Cysts Form In Vivo

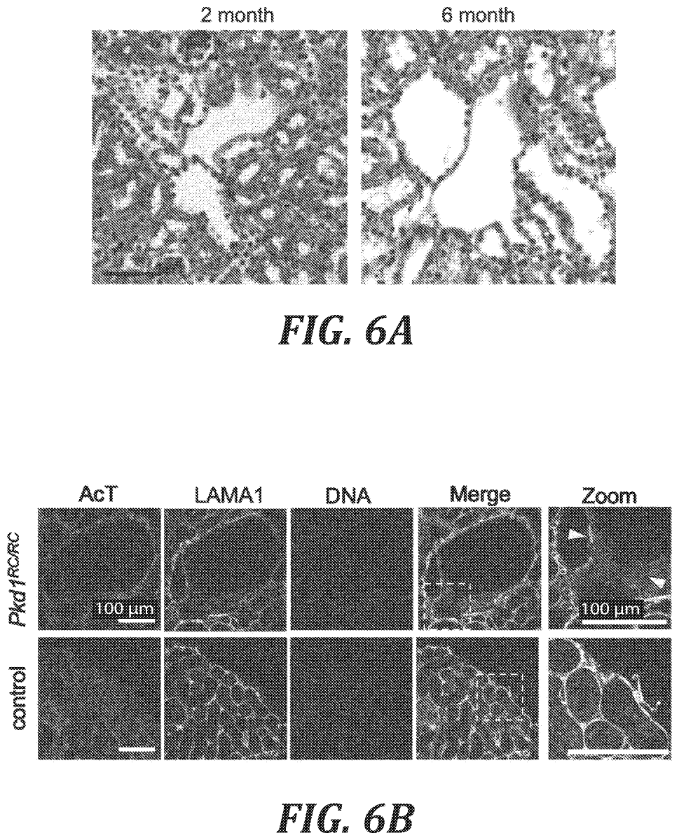

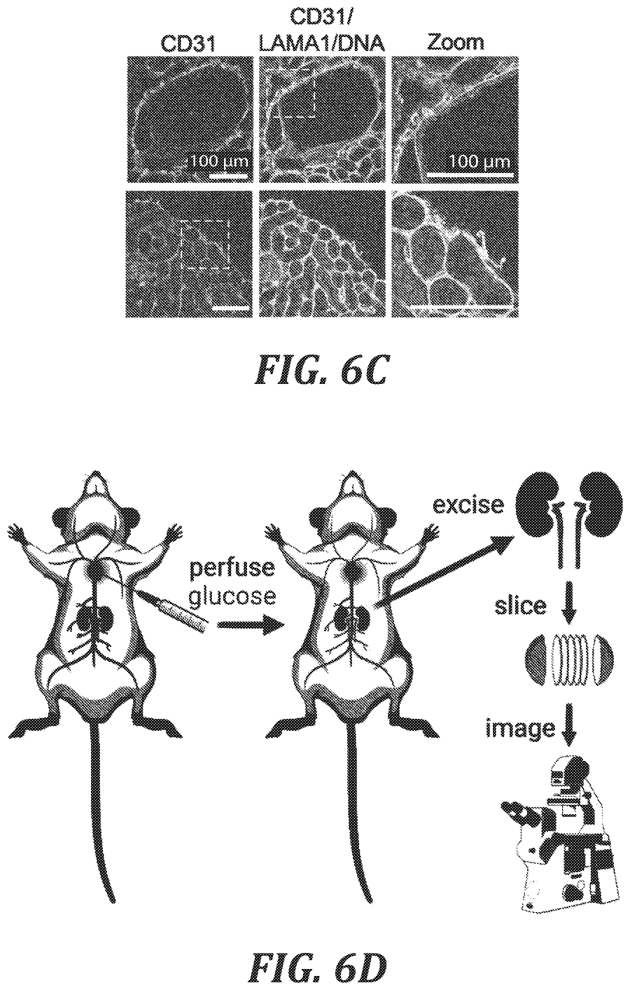

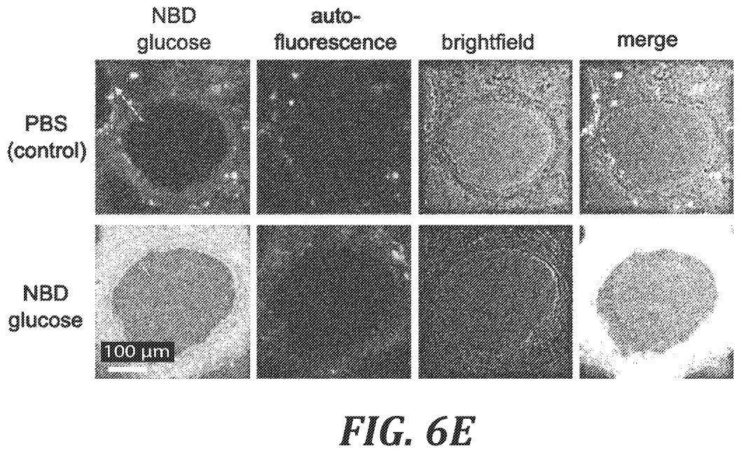

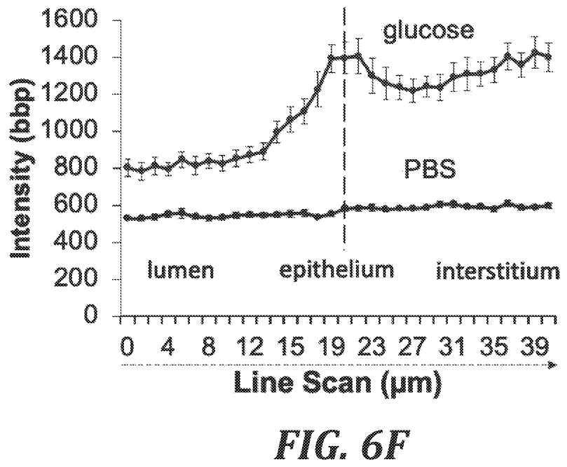

Microcysts smaller than 1 mm diameter and undetectable by magnetic resonance imaging are numerous in kidney sections from patients with early stages of PKD, and have been proposed to form as focal outpouchings of tubular epithelium. If such an outpouching remained connected to a small segment of the original tubule via apical junctions, it could accumulate fluid through tubular reabsorption. To investigate this possibility in vivo, microcysts in the Pkd1^{RC/RC} mouse were analyzed. These mice have a hypomorphic Pkd1 gene mutation orthologous to patient disease variant PKD1 p. R3277C, and manifest a slowly progressive PKD during adulthood over a period of several months. Histology sections and confocal images of 2-month-old mouse tissue revealed continuous basement membranes between tubules and microcysts, consistent with the possibility that microcysts form from tubular outpouchings that remain capable of absorption through the wall of the neighboring tubule (FIG. 6A, FIG. 6B). While much of these microcysts remained tightly associated with peritubular capillaries, suggesting that they continue to reabsorb, portions of the epithelium appeared to have detached from the endothelium, resulting in areas of fluid accumulation or interstitial expansion (FIG. 6B, FIG. 6C).

To determine whether PKD cysts absorbed glucose in vivo, a methodology was devised to inject mice with NBD glucose and immediately retrieve their kidneys (FIG. 6D). Fluorescence microscopy analysis of kidney tissue sections revealed that cyst-lining epithelia and the surrounding interstitium readily took up NBD glucose (FIG. 6E, FIG. 6F). Thus, cysts remained absorptive in vivo and PKD kidneys as a whole readily accumulated glucose.

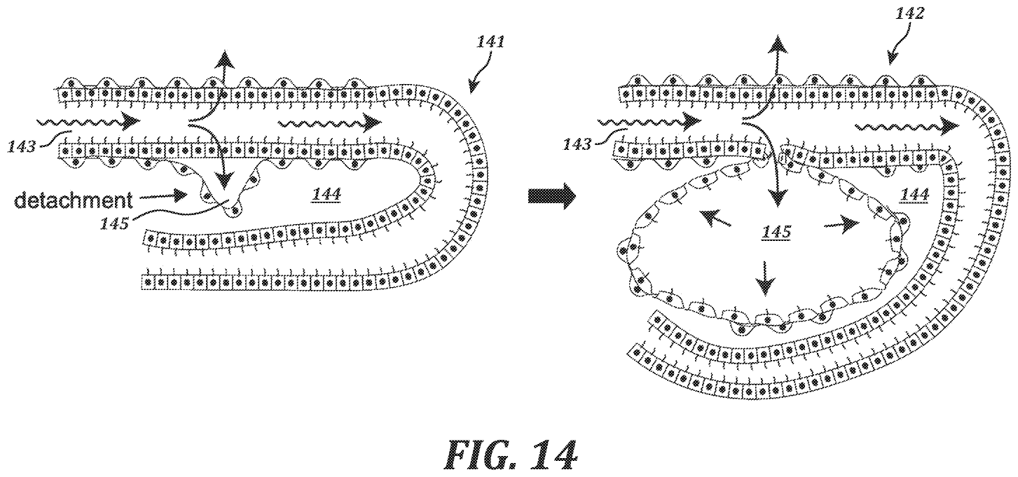

Without wishing to be bound by any particular theory, the preceding suggested a possible model for cyst formation in vivo (FIG. 14). Absorption of glucose through the apical surface of the tubular epithelium is followed by water along the osmotic gradient via paracellular or transcellular routes to maintain balanced concentrations on either side of the epithelium. There is a lack of appropriate outlet for this absorptive activity, creating a pressure within the interstitium and leading to its detachment from neighboring tubules, which undergo deformation and expansion to fill the resultant interstitial space. This process continues as the cyst grows, and may be exacerbated by the gradual loss or detachment of associated peritubular capillaries (which reduces the absorptive sink), and by growth of interstitial mesenchymal stromal cells, which provide a scaffold and synthesize extracellular matrix to accommodate the expanding epithelium.

### Discussion

Coupling the structural and functional characteristics of organoids with the controlled, microfluidic microenvironments of organ-on-a-chip devices is a promising approach to in vitro disease modeling. This example combines CRISPR-Cas9 gene editing to reconstitute disease phenotype with organoid-on-a-chip technology to understand the effect of flow, which is difficult to assess in vivo (where it is constant) and has hitherto been absent from kidney organoid models at physiological strength. The ‘human kidney organoid on a chip’ microphysiological system described herein incorporates organoids with PKD mutations in a wide-channel format, which allows liquid to flow over the organoids. The system can be readily assembled from commercially available components, and produces a shear stress associated with the physiological range found in human kidney tubules. This is ˜6-fold greater than the maximum rate of 0.035 dyn/cm² used in a previous kidney organoid-on-a-chip device, a shear stress that was nevertheless sufficient to stimulate expansion of vasculature within the device when compared to static conditions.

Interestingly, a static module using the same chip that is capable of diffusion from a syringe reservoir has also been described herein. This enables the person having skill in the art to distinguish the effects of flow from those of exposure to fluid volume and mass of reabsorbable solute, which is difficult to achieve in conventional systems with limited diffusion such as by tightly connecting a reservoir to a Luer lock syringe. The discovery that volume can partially substitute for flow is a novel finding compared with previous studies which were unable to observe this effect. In addition to volume, hydrostatic pressure is increased in the diffusive static condition, which may play a role in PKD phenotype. Of note, cysts in the diffusive static condition did not exhibit the dramatic oscillations in size observed under flow, indicating roles for flow-induced mechanoregulation that cannot be readily replicated by diffusion effects, for example, involving stretch-activated ion channels.

The example findings indicate that flow, volume, and solute concentrations are positive regulators of cyst expansion. Cystogenesis can be enhanced through mechanisms of tubular absorption and glucose transport. However, as peripheral epithelia in organoids face outwards towards the media, the net result is for the apical surface to be in contact with the directional flow, similar to the epithelium of a tubule in vivo. Without wishing to be bound by any particular theory, the observation that PKD cysts can form inside-out, such that the secretion (basolateral-to-apical transport) would occur in the opposite direction from cysts in vivo, argues against secretion as the critical driver of cystogenesis in this model system. The experiments in animals also demonstrate that kidney cysts remain reabsorptive even in advanced PKD. These findings are consistent with macropuncture studies showing that wall pressures inside PKD cysts in vivo resemble their originating nephron segments, and studies of excised cysts in vitro, which demonstrate that the epithelium is slowly expanding and absorptive under steady-state conditions. In a clinical analysis, patients with ADPKD demonstrated lower excretion of renally secreted solutes, rather than higher levels of secretion. Without wishing to be bound by any particular theory, drugs that activate CFTR, which is hypothesized to drive a secretory phenotype in PKD, have shown promise in treating PKD in mice, rather than exacerbating the disease, which is also inconsistent with a secretory hypothesis. However, this is not to say that secretion cannot be a causative mechanism in PKD cystogenesis, but rather that absorption can also play a role.

It was observed that transfer of PKD organoids from adherent cultures into suspension cultures was associated with dramatically increased rates of cystogenesis, and the example findings add greatly to the understanding of this phenomenon. Upon release from the underlying substratum, the peripheral organoid epithelium grows out and envelops the rest of the organoid. This forms an enclosed, outwards-facing structure in an ideal conformation to absorb fluid from the surrounding media and expand into a cyst. Although differences in the levels of SGLT2 were not detected, differences may exist in SGLT2 activity, or in the levels or activity of other transporters involved in absorption, resulting in increased absorptive flux in PKD epithelia, compared to non-PKD. Alternatively, there might exist a difference in the pliability of PKD epithelia versus non-PKD epithelia undergoing equivalent levels of absorptive flux. Without wishing to be bound by any particular theory, it is noted that polycystin-2 is a non-selective cation channel expressed at the apical plasma membrane, which could conceivably play a role in transporter function and reabsorption. The polycystin complex may also possess force- or pressure-sensitive mechanoreceptor properties, which could regulate the epithelial response to fluid influx.

Although a direct role for glucose absorption in driving cyst expansion may be favored, glucose transport could also function separately of water transport to impact cyst formation, for instance by altering mitochondrial metabolism or signaling changes to the actin cytoskeleton, which could promote cystogenesis regardless of which direction the cells face. Of note, cysts form not only in the proximal tubules that are primarily responsible for glucose reabsorption, but also in the collecting ducts, where they can reach very large sizes. As cysts originate from these very different epithelial cell types, the process of cystogenesis is not likely to be explained by a simple absorption/secretion ratio for any one solute. In implementations of the PKD organoid system, collecting ducts are incorporated, as some cell lineages are relevant to PKD cystogenesis but do not mature in human kidney organoid cultures.

In this example, the organoid phenotype is limited to biallelic mutants, in which disease processes are greatly accelerated. However, germline mutations in PKD patients are monoallelic, and phenotypes take decades to develop, possibly due to developing ‘second hit’ somatic mutations in the second allele. The example system involving biallelic mutants may more closely phenocopy early-onset autosomal recessive PKD than late-onset autosomal dominant PKD, which can be considered when extrapolating some findings into a clinical context. Generation of well-controlled allelic series of PKD organoids, together with methodologies to model the acquisition of somatic mutations, can be implemented to produce human organoid models with greater fidelity to autosomal dominant PKD.

Canagliflozin (Invokana), an inhibitor of SGLT2, has recently been approved for the treatment of type II diabetes, and appears to have a protective effect in the kidneys. Clinical studies and other efforts have not tried to use SGLT inhibitors in patients with PKD. These example findings suggest that blocking SGLT activity could reduce proximal tubule cysts by preventing glucose reabsorption. However, this may also expose the collecting ducts downstream to higher glucose concentrations.

In summary, a microfluidic kidney organoid module is provided that enables detailed studies of renal tubular absorption and PKD cyst growth. The cyst-lining epithelium in this system is exposed to flow in a mirror image of the nephron structure in vivo. Using this system, glucose levels and its transport into cyst structures are identified as a driver of cystic expansion in proximal nephron-like structures. Therapeutics that modulate reabsorption may therefore be beneficial in reducing cyst growth in specific nephron segments, with relevance for future PKD clinical trials.

### Methods

CRISPR-based truncating mutations to PKD1, PKD2. Constructs encoding green fluorescent protein-tagged Cas9 and a guide RNA targeting the first exon of PKD2 (5′-GCGTGGAGCCGCGATAACCC-3′; SEQ ID NO. 1) or the thirty-seventh exon of PKD1 (5′-CGCACGTGTGCCCCGCGTA-3′; SEQ ID NO. 2) were transiently transfected into undifferentiated induced pluripotent stem (iPS) cells, and green fluorescent protein-expressing cells were isolated by flow cytometric sorting, clonally expanded, and screened for clones with loss-of function indels using sequencing of the genomic DNA.

Kidney organoid differentiation. Work with hPSC was performed under the approval and auspices of the University of Washington Embryonic Stem Cell Research Oversight Committee. Specific cell lines used in this study are described below and are sourced from commercially available hPSC obtained with informed consent. hPSC stocks were maintained in mTeSR1 media with daily media changes and weekly passaging using Accutase or ReLeSR (STEMCELL Technologies, Vancouver). 5,000-20,000 hPSCs per well were placed in each 24-well plate pre-coated with 300 μL of DMEM-F12 containing 0.2 mg/mL Matrigel and sandwiched the following day with 0.2 mg/mL Matrigel in mTeSR1 (STEMCELL Technologies, Vancouver) to produce scattered, isolated spheroid colonies. 48 hrs after sandwiching, hPSC spheroids were treated with 12 μM CHIR99021 (Tocris Bioscience) for 36 h, then changed to RB (Advanced RPMI+1× Glutamax+1×B27 Supplement, all from Thermo Fisher Scientific) after 48 hours, and replaced with fresh RB every 3 days thereafter.

Organoid perfusion in microfluidic chip. Ibidi μ-Slide VI0.4 were coated with 3.0% Reduced Growth Factor Geltrex (Life Technologies) and left at 37° C. overnight to solidify. Kidney organoids (21-40 d) were picked from adhered culture plates, pipetted into the slide channels (2-3 per channel) with RB, and left for 24 hrs at 37° C. to attach. Organoids were distributed randomly within the channel. For the fluidic condition, 60 mL syringes filled with RB were attached to channels using clear tubing (Cole-Parmer, 0.02″ ID, 0.083″ OD). A clamp was used to close off the tubing, and the media in the syringe was changed to 25 mL RB+36.5 μM 2-NBD-Glucose fluorescent glucose (Abcam ab146200). A Harvard Apparatus syringe infusion pump was used to direct media flow into microfluidic chip at 160 μL/min (0.2 dynes/cm2). Media was collected at the outlet and filtered for repeated use. For the static condition, a 25 mL syringe containing RB was attached to the channel using wide clear tubing (Cole-Parmer, 0.125″ ID, 0.188″ OD). The syringe was detached momentarily, the plunger removed, and the open syringe reattached and filled slowly with 25 mL RB+36.5 μM 2-NBD-Glucose. From this point on, diffusion of the fluorescent glucose began from the open syringe into the channel via the tubing. Alternatively, NDB-glucose was substituted with food dye (invert sugar, 360 g/mol), or alternatively the organoids were perfused with media in the absence of any additives.

Image/video collection. Image collection was performed on a Nikon Ti Live-Cell Inverted Widefield microscope inside of an incubated live imaging chamber supplemented with 5% carbon dioxide. Experiments in microfluidic devices were recorded for 6 hours. During this time, cysts changed in volume (grew and shrank) and in some cases were destroyed due to bubbles arising in the tubing. Cyst growth rate in microfluidic devices was therefore calculated on an individual basis, when each cyst reached its maximal volume, which varied for each sample from 1 hour to 5 hours after the start of the experiment. For longer-term experiments conducted in static 96-well cultures, organoids were imaged at regular intervals (typically 24 hours) and analyzed at the endpoint indicated in the figure graphs. Phase contrast and GFP (200 ms exposure) images were taken every 5 minutes for a maximum of 12 hours. Images of fixed samples were collected on a Nikon AIR point scanning confocal microscope.

Animal Studies. Kidney tissue from Pkd1^{RC/RC} mice maintained in C57BL/6J background (gift of Mayo Clinic Translational PKD Center) and C57BL/6J controls were utilized. In order to investigate the process of cystogenesis, younger Pkd1^{RC/RC} mice 6-7 weeks of age, along with wild-type C57BL/6J mice of the same age were used. Kidneys were harvested after systemic perfusion with ice-cold PBS, followed by fixation with paraformaldehyde fixative and immersion in 18-30% sucrose at 4° C. overnight. Tissues were embedded and frozen in optimal cutting temperature compound (OCT, Sakura Finetek, Torrance, CA). Cryostat-cut mouse kidney sections (5-10 μm) were stained for acetylated α-tubulin, laminin-1, and CD31 (see “Immunostaining” for primary antibodies and dilutions).

For perfusion experiments, NBD Glucose was freshly dissolved in PBS to a concentration of 1 mM. Freshly sacrificed Pkd1^{RC/RC} mice (>8 months old) were incised through the chest and nicked at the vena cava with a 27-gauge needle. Keeping pressure on the vena cava, mice were perfused systemically through the heart with a syringe containing 10 mL of PBS, followed by a second syringe containing 5 mL of either PBS alone (control) or PBS+1 mM NBD-Glucose. Kidneys were harvested immediately and embedded fresh without fixation or sucrose equilibration in OCT. Cryostat-cut mouse kidney sections (20 μm) were mounted in OCT and imaged on a confocal microscope with 10× objective. All animal studies were conducted in accordance with all relevant ethical regulations under protocols approved by the Institutional Animal Care and Use Committee at the University of Washington in Seattle. Mice were maintained on a standard diet under standard pathogen-free housing conditions, with food and water freely available.

Immunostaining. Immunostaining followed by confocal microscopy was used to localize various proteins and transporters in the cysts and organoids. Prior to staining, an equal volume of 8% paraformaldehyde was added to the culture media (4% final concentration) for 15 mins at room temperature. After fixing, samples were washed in PBS, blocked in 5% donkey serum (Millipore)/0.3% Triton-X-100/PBS, incubated overnight in 1% bovine serum albumin/0.3% Triton-X-100/10M CaCl₂/PBS with primary antibodies, washed, incubated with Alexa-Fluor secondary antibodies (Invitrogen), washed and imaged. Primary antibodies or labels include acetylated α-tubulin (Sigma T7451, 1:5,000), ZO-1 (Invitrogen 61-7300, 1:200), Biotinylated LTL (Vector Labs B-1325, 1:500), E-Cadherin (Abcam ab11512, 1:500), SGLT2 (Abcam ab37296, 1:100), laminin-1 (Sigma L9393, 1:50), alpha smooth muscle actin (Sigma A2547, 1:500), CD31 (BD Biosciences 557355, 1:300). Fluorescence images were captured using a Nikon AIR inverted confocal microscope with objectives ranging from 10× to 60×.

Statistical Analysis. Experiments were performed using a cohort of PKD hPSC, generated and characterized as described previously, including three PKD2^{-/-} hPSC lines and three isogenic control lines that were subjected to CRISPR mutagenesis but were found to be unmodified at the targeted locus by Sanger sequencing of each allele and immunoblot. Altogether these represented two distinct genetic backgrounds, genders, and cell types: (i) male WTC11 iPS cells (Coriell Institute Biobank, GM25256, two isogenic pairs) and (ii) female H9 ES cells (WiCell, Madison Wisconsin, WA09, one isogenic pair). Quantification was performed on data obtained from experiments performed on controls and treatment conditions side by side on at least three different occasions or cell lines (biological replicates). Error bars are mean±standard error (s.e.m.). Statistical analyses were performed using GraphPad Prism Software. To test significance, p values were calculated using two-tailed, unpaired or paired t-test (as appropriate to the experiment) with Welch's correction (unequal variances). For multiple comparisons, standard ANOVA was used. Statistical significance was defined as p<0.05. Exact or approximate p values are provided in the figure legends in experiments that showed statistical significance. For traces of cysts over time, the least squares progression model was applied to fit the data to lines in GraphPad Prism. Line scans of equal length were averaged from multiple images and structures based on raw data intensity values in the GFP channel. Lines were drawn transecting representative regions of each structure (e.g., avoiding heterogeneities, brightness artifacts, or areas where cysts and organoids overlapped), placed such that the first half of each line represented the background in the image. The intensity of each point (pixel) along the line was then averaged for all of the lines, producing an averaged line scan with error measurements. Arrows are provided in representative images showing the direction and length of the line scans used to quantify the data. Unless otherwise noted, raw intensity values (bytes per pixel) were used without background subtraction.

Hydrostatic pressure calculation. The calculation was performed according to the steps shown at FIG. 15. The height from channel to top of media in reservoir was measured to be: Static 1 mL: ˜12 cm; Static 25 mL: ˜20 cm. This amounted to a total difference in pressure of (14.7-8.8=5.9) mmHg.

Incorporated by reference, with variations and pseudogenes: Gene: PKD1 (ENSG00000008710); Gene: PKD2 (ENSG00000118762).

### Definitions

Specific elements of any foregoing embodiments can be combined or substituted for elements in other embodiments. Moreover, the inclusion of specific elements in at least some of these embodiments may be optional, wherein further embodiments may include one or more embodiments that specifically exclude one or more of these specific elements. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

As used herein, the term “individual,” “subject,” or “patient,” used interchangeably, refers to any animal, including mammals, preferably mice, rats, other rodents, rabbits, dogs, cats, swine, cattle, sheep, horses, or primates, and most preferably humans.

One letter codes for amino acids are used herein. For example, alanine is A, arginine is R, asparagine is N, aspartic acid is D, asparagine or aspartic acid is B, cysteine is C, glutamic acid is E, glutamine is Q, glutamine or glutamic acid is Z, glycine is G, histidine is H, isoleucine is I, leucine is L, lysine is K, methionine is M, phenylalanine is F, proline is P, serine is S, threonine is T, tryptophan is W, tyrosine is Y, valine is V.

As used herein, the term “organs” refers to a group of tissues in a living organism that have been adapted to perform a specific function.

As used herein, the term “protein” refers to a polypeptide and/or any of various naturally occurring substances that are comprised of amino-acid residues joined by peptide bonds, contain the elements carbon, hydrogen, nitrogen, oxygen, usually sulfur, and occasionally other elements (such as phosphorus or iron), and include many essential biological compounds (such as enzymes, hormones, or antibodies).

As used herein, the term “small molecule” refers to a low molecular weight (<2000 daltons) organic compound that may help regulate a biological process, with a size on the order of 1 nm.

As used herein, the term “therapeutic agent” refers to a substance capable of producing a therapeutic effect in a disease state.

As used herein, the term “tissue” refers to an aggregate of similar cells and cell products forming a structural material with a specific function, in a multicellular organism.

As used herein, “PKD genotype” refers to a genotype associated with PKD or a model for PKD.

As used herein and unless otherwise indicated, the terms “a” and “an” are taken to mean “one”. “at least one” or “one or more”. Unless otherwise required by context, singular terms used herein shall include pluralities and plural terms shall include the singular.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present disclosure. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

All of the references cited herein are incorporated by reference, and reference to such references is not to be taken as admission that such references form any part of the body of prior art. Aspects of the disclosure can be modified, if necessary, to employ the systems, functions, and concepts of the above references and application to provide yet further embodiments of the disclosure. These and other changes can be made to the disclosure in light of the detailed description.

### Non-Limiting Embodiments

While general features of the disclosure are described and shown and particular features of the disclosure are set forth in the claims, the following non-limiting embodiments relate to features, and combinations of features, that are explicitly envisioned as being part of the disclosure. The following non-limiting Embodiments, and features thereof, are modular and can be combined with each other in any number, order, or combination to form a new non-limiting Embodiment, which can itself be further combined with other non-limiting Embodiments and features thereof.

Embodiment 1. A method for characterizing PKD in vitro, the method comprising: culturing a GM human kidney organoid within a flow device including a channel with a functionalized site configured for cell culture and an inlet and an outlet configured for flow of a fluid at the functionalized site for formation of a PKD cyst; contacting the PKD cyst with the fluid including a property; and measuring a response of the PKD cyst to the property of the fluid.

Embodiment 2. The method of any other Embodiment, wherein the human kidney organoid has a genetic modification that is associated with PKD.

Embodiment 3. The method of any other Embodiment, further comprising: creating a PKD genotype in a human stem cell, wherein the PKD genotype comprises a loss-of-function mutation; and differentiating the human stem cell into the GM human kidney organoid.

Embodiment 4. The method of any other Embodiment, further comprising: genetically deactivating a PKD2 gene in the human stem cell and/or the GM human kidney organoid to produce a PKD2 negative human stem cell and/or a PKD2 negative GM human kidney organoid; and/or genetically deactivating a PKD1 gene in the human stem cell and/or the GM human kidney organoid to produce a PKD1 negative human stem cell and/or a PKD1 negative GM human kidney organoid.

Embodiment 5. The method of any other Embodiment, wherein the characterization comprises: determining a mechanism of PKD cyst formation, expansion, and/or contraction in response to the property of the fluid.

Embodiment 6. The method of any other Embodiment, further comprising: determining whether the PKD cyst absorbs glucose; determining whether glucose absorption increases PKD cyst formation; and/or determining a polarization of the PKD cyst.

Embodiment 7. The method of any other Embodiment, further comprising: controlling a volume, a solute concentration, and/or a flow rate of the fluid as the property of the fluid; and measuring the response of the PKD cyst to the volume, the solute concentration, and/or the flow rate of the fluid.

Embodiment 8. The method of any other Embodiment, wherein a flow of the fluid corresponds with formation and/or expansion of the PKD cyst.

Embodiment 9. The method of any other Embodiment, wherein the inlet is fluidly connected to the outlet and the fluid flows from the inlet through the channel toward the outlet according to the flow rate and flows over the PKD cyst of the GM human kidney organoid.

Embodiment 10. The method of any other Embodiment, further comprising: contacting the PKD cyst with a glucose transport inhibitor to determine an effect of glucose transport inhibition on PKD cyst formation, expansion. and/or contraction.

Embodiment 11. The method of any other Embodiment, wherein the glucose transport inhibitor comprises phloretin, phloridzin, and/or dapagliflozin.

Embodiment 12. A method for formation of a PKD cyst of a GM human kidney organoid for characterizing PKD in vitro, the method comprising: culturing the GM human kidney organoid within a flow device having a channel with a functionalized site configured for cell culture and an inlet and an outlet configured for flow of a fluid at the functionalized site; and contacting the PKD cyst with a flow of the fluid.

Embodiment 13. The method of any other Embodiment, wherein the human kidney organoid has a genetic modification that is associated with PKD.

Embodiment 14. The method of any other Embodiment, further comprising: contacting the PKD cyst with glucose thereby increasing formation of the PKD cyst.

Embodiment 15. The method of any other Embodiment, wherein the characterization comprises: determining a mechanism of PKD cyst formation, expansion, and/or contraction in response to the fluid.

Embodiment 16. The method of any other Embodiment, further comprising: controlling a volume, a solute concentration, and/or a flow rate of the fluid as the property of the fluid; and determining the response of the PKD cyst to the volume, the solute concentration, and/or the flow rate of the fluid.

Embodiment 17. The method of any other Embodiment, wherein a flow of the fluid corresponds with formation of the PKD cyst.

Embodiment 18. The method of any other Embodiment, wherein the inlet is fluidly connected to the outlet and the fluid flows from the inlet through the channel toward the outlet according to the flow rate and flows over the PKD cyst of the GM human kidney organoid.

Embodiment 19. A microfluidic system for characterization of PKD in vitro, the microfluidic system comprising: a flow device having a channel with a functionalized site configured for cell culture and an inlet and an outlet configured for flow of a fluid at the functionalized site, wherein the inlet is fluidly connected with the outlet via the channel.

Embodiment 20. The microfluidic system of any other Embodiment, further comprising a GM human pluripotent stem cell or a GM human kidney organoid configured for a PKD cyst and having a genetic modification that is associated with PKD.

Embodiment 21. The microfluidic system of any other Embodiment, wherein the genetic modification that is associated with PKD comprises a genetically deactivated PKD2 gene and/or a genetically deactivated PKD1 gene.

Embodiment 22. The microfluidic system of any other Embodiment, wherein the functionalized site comprises an extracellular matrix (ECM) for cell culture.

While illustrative embodiments have been illustrated and described, it will be appreciated that various changes can be made therein without departing from the spirit and scope of the invention.

1. A method for characterizing polycystic kidney disease (PKD) in vitro, the method comprising:
culturing a genetically modified (GM) human kidney organoid within a flow device including a channel with a functionalized site configured for cell culture and an inlet and an outlet configured for flow of a fluid at the functionalized site for formation of a PKD cyst;
contacting the PKD cyst with the fluid including a property; and
measuring a response of the PKD cyst to the property of the fluid.

2. The method of claim 1, wherein the human kidney organoid has a genetic modification that is associated with PKD.

3. The method of one of claim 1, further comprising:
creating a PKD genotype in a human stem cell, wherein the PKD genotype comprises a loss-of-function mutation; and
differentiating the human stem cell into the GM human kidney organoid.

4. The method of one of claim 3, further comprising:
genetically deactivating a PKD2 gene in the human stem cell and/or the GM human kidney organoid to produce a PKD2 negative human stem cell and/or a PKD2 negative GM human kidney organoid; and/or
genetically deactivating a PKD1 gene in the human stem cell and/or the GM human kidney organoid to produce a PKD1 negative human stem cell and/or a PKD1 negative GM human kidney organoid.

5. The method of one of claim 1, wherein the characterization comprises:
determining a mechanism of PKD cyst formation, expansion, and/or contraction in response to the property of the fluid.

6. The method of one of claim 1, further comprising:
determining whether the PKD cyst absorbs glucose;
determining whether glucose absorption increases PKD cyst formation; and/or
determining a polarization of the PKD cyst.

7. The method of one of claim 1, further comprising:
controlling a volume, a solute concentration, and/or a flow rate of the fluid as the property of the fluid; and
measuring the response of the PKD cyst to the volume, the solute concentration, and/or the flow rate of the fluid.

8. The method of one of claim 1, wherein a flow of the fluid corresponds with formation and/or expansion of the PKD cyst.

9. The method of one of claim 1, wherein the inlet is fluidly connected to the outlet and the fluid flows from the inlet through the channel toward the outlet according to the flow rate and flows over the PKD cyst of the GM human kidney organoid.

10. The method of one of claim 1, further comprising:
contacting the PKD cyst with a glucose transport inhibitor to determine an effect of glucose transport inhibition on PKD cyst formation, expansion, and/or contraction.

11. The method of one of claim 10, wherein the glucose transport inhibitor comprises phloretin, phloridzin, and/or dapagliflozin.

12. A method for formation of a PKD cyst of a GM human kidney organoid for characterizing PKD in vitro, the method comprising:
culturing the GM human kidney organoid within a flow device, wherein the flow device has a channel with a functionalized site configured for cell culture and an inlet and an outlet configured for flow of a fluid at the functionalized site, wherein the inlet is fluidly connected to the outlet for a fluid to flow from the inlet through the channel towards the outlet according to a flow rate, and wherein the fluid flows over the functionalized site; and
contacting the PKD cyst with a flow of the fluid, wherein a flow of the fluid corresponds with formation of the PKD cyst.

13. The method of claim 12, wherein the human kidney organoid has a genetic modification that is associated with PKD.

14. The method of one of claim 12, further comprising:
contacting the PKD cyst with glucose thereby increasing formation of the PKD cyst.

15. The method of one of claim 12, wherein the characterization comprises:
determining a mechanism of PKD cyst formation, expansion, and/or contraction in response to the fluid.

16. The method of one of claim 12, further comprising:
controlling a volume, a solute concentration, and/or a flow rate of the fluid as the property of the fluid; and
determining the response of the PKD cyst to the volume, the solute concentration, and/or the flow rate of the fluid.

17-18. (canceled)

19. A microfluidic system for characterization of PKD in vitro, the microfluidic system comprising:
a flow device having a channel with a functionalized site configured for cell culture and an inlet and an outlet configured for flow of a fluid at the functionalized site, wherein the inlet is fluidly connected with the outlet via the channel.

20. The microfluidic system of claim 19, further comprising a GM human pluripotent stem cell or a GM human kidney organoid configured for a PKD cyst and having a genetic modification that is associated with PKD.

21. The microfluidic system of claim 20, wherein the genetic modification that is associated with PKD comprises a genetically deactivated PKD2 gene and/or a genetically deactivated PKD1 gene.

22. The microfluidic system of one of claim 19, wherein the functionalized site comprises an extracellular matrix (ECM) for cell culture.
